(19) United States
(12) Patent Application Publication
(10) Pub. No.: US 2017/0029904 A1
(43) Pub. Date: Feb. 2, 2017

(54) CLASSIFICATION OF MYC-DRIVEN B-CELL LYMPHOMAS

(71) Applicants: The Brigham and Women's Hospital, Inc., Boston, MA (US); TRUSTEES OF BOSTON UNIVERSITY, Boston, MA (US)

(72) Inventors: Scott J. Rodig, Westwood, MA (US); Christopher Daniel Carey, Boston, MA (US); Margaret A. Shipp, Wellesley, MA (US); Stefano Monti, Somerville, MA (US); Daniel Gusenleitner, Allston, MA (US); Bjoern Chapuy, Boston, MA (US)

(21) Appl. No.: 15/302,298

(22) PCT Filed: Apr. 7, 2015
(86) PCT No.: PCT/US15/24724

§ 371 Date: Oct. 6, 2016

Related U.S. Application Data
(60) Provisional application No. 61/976,298, filed on Apr. 7, 2014.

(51) Int. Cl. C12Q 1/68; C07K 16/28; A61K 39/395; A61K 31/704; A61K 31/475; A61K 31/573; G06F 19/12; A61K 31/664

(52) U.S. Cl. CPC C12Q 1/6886; G06F 19/12; C07K 16/2887; A61K 31/664; A61K 31/704; A61K 31/475; A61K 31/573; A61K 39/3955; C12Q 2600/158; C12Q 2600/118; C12Q 2600/112; C07K 2317/24

(57) ABSTRACT

Methods for diagnosing Burkitt lymphoma (BL) and diffuse large B-cell lymphoma (DLBCL) based on a diagnostic score, as well as determining MYC activity levels and selecting treatments based on a MYC activity score.

### CLAIM OF PRIORITY

This application claims the benefit of U.S. Provisional Application Ser. No. 61/976,298, filed on Apr. 7, 2014. The entire contents of the foregoing are incorporated herein by reference.

### FEDERALLY SPONSORED RESEARCH OR DEVELOPMENT

This invention was made with Government support under Grant No. P01CA092625 awarded by the National Institutes of Health. The Government has certain rights in the invention.

### TECHNICAL FIELD

Described are methods for diagnosing Burkitt lymphoma (BL) and diffuse large B-cell lymphoma (DLBCL) based on a diagnostic score, as well as determining MYC activity levels and selecting treatments based on a MYC activity score.

### BACKGROUND

Burkitt lymphoma (BL) and diffuse large B-cell lymphoma (DLBCL) are aggressive tumors of mature B-cells that are distinguished by a combination of histomorphologic, phenotypic, and genetic features. A subset of B-cell lymphomas, however, has one or more characteristics that overlap BL and DLBCL, and are categorized as B-cell lymphoma unclassifiable, with features intermediate between BL and DLBCL (BCL-U).

### SUMMARY

Molecular analyses support the concept that there is a biological continuum between BL and DLBCL that includes variable activity of MYC, an oncoprotein once thought to be only associated with BL, but now recognized as a major predictor of survival among patients with DLBCL treated with R-CHOP. We tested whether a targeted expression profiling panel could be used to categorize tumors as BL and DLBCL, resolve the molecular heterogeneity of BCL-U, and capture MYC activity using RNA from formalin-fixed paraffin embedded biopsies. A diagnostic molecular classifier accurately predicted pathological diagnoses of BL and DLBCL, and provided more objective sub-classification for a subset of BCL-U and genetic “double-hit” lymphomas as molecular BL or DLBCL. A molecular classifier of MYC activity correlated with MYC IHC and stratified patients with primary DLBCL treated with R-CHOP into high- and low-risk groups. These results establish a framework for classifying and stratifying MYC-driven, aggressive B-cell lymphomas based upon quantitative molecular profiling that is applicable to fixed biopsy specimens.

Thus, provided herein are methods for diagnosing a subject who has a B-cell lymphoma, e.g., B-cell lymphoma unclassifiable (BCL-U), as having Burkitt lymphoma (BL) or diffuse large B-cell lymphoma (DLBCL). The methods include obtaining a sample comprising cells from the B-cell lymphoma in a subject; determining levels of mRNA for diagnostic signature genes in the cells, wherein the diagnostic signature genes comprise STRBP, PRKAR2B, E2F2, LZTS1, CDC25A, TCF3, RANBP1, DLEU1, PAICS, DNMT3B, PPAT, KIAA0101, PYCR1, CD10, NME1, FAM216A/C12ORF24, BMP7, BCL2, CD44, p50 (NFKB1), and BCL2A; calculating a diagnostic score based on the mRNA levels; and diagnosing DLBCL when the diagnostic score is below a first threshold, diagnosing BL when the diagnostic scores is above a second threshold that is higher than the first threshold, and diagnosing intermediate B-cell lymphoma when the diagnostic score is between the first and second thresholds.

Also provided herein are methods for predicting response to treatment in a subject who has a B-cell lymphoma, e.g., diffuse large B-cell lymphoma (DLBCL). The methods include obtaining a sample comprising cells from a B-cell lymphoma in a subject; determining levels of mRNA for MYC activity signature genes in the cells, wherein the diagnostic signature genes comprise MYC, SRM, AKAP1, NME1, FBL, RFC3, TCL1A, POLD2, RANBP1, GEMIN4, MRPS34, DHX33, PPRC1, PPAT, FAM216A/C12ORF24, PAICS, UCHL3, NOLC1, KIAA0226L, PRMT1, LDHB, TRAP1, AHCY, LRP8, EBNA1BP2, CDK4, ETFA, UCK2, CTPS, GOT2, FAM211A/C17ORF76, TMEM97, RRS1, DDX21, PHB2, WDR3, KIAA0101, FASN, SAMD13, CDC25A, LYAR, CAD, APEX1, NOP2, PHB, SSBP1, MRPS2, CIRH1A, SLC16A1, BUB1B, APITD1, NCL, DLEU1, PCDH9, IGFBP2, TDO2, SLC12A8, P2RY12, TMEM119, SHISA8, and SLAMF1; calculating a MYC activity score based on the mRNA levels; and predicting response to treatment based on the MYC activity score.

In some embodiments, a MYC activity score above a threshold level indicates that the subject is not likely to respond to the treatment, and a MYC activity score below the threshold level indicates that the subject is likely to respond to the treatment.

In some embodiments, the methods described herein include selecting a subject who has a MYC activity score below the threshold level, and optionally administering the treatment to the subject.

In some embodiments of the methods described herein, the treatment is the R-CHOP (rituximab, cyclophosphamide, doxorubicin, vincristine, and prednisone) regimen.

In addition, provided herein are methods for selecting, excluding or stratifying a subject for a clinical trial. The methods include one or both of: (i) determining a diagnostic score for the subject by obtaining a sample comprising cells from the B-cell lymphoma in the subject; determining levels of mRNA for diagnostic signature genes in the cells, wherein the diagnostic signature genes comprise STRBP, PRKAR2B, E2F2, LZTS1, CDC25A, TCF3, RANBP1, DLEU1, PAICS, DNMT3B, PPAT, KIAA0101, PYCR1, CD10, NME1, FAM216A/C12ORF24, BMP7, BCL2, CD44, p50 (NFKB1), and BCL2A; calculating a diagnostic score based on the mRNA levels; and/or (ii) determining a MYC activity score method for the subject by obtaining a sample comprising cells from a B-cell lymphoma in a subject; determining levels of mRNA for MYC activity signature genes in the cells, wherein the diagnostic signature genes comprise MYC, SRM, AKAP1, NME1, FBL, RFC3, TCL1A, POLD2, RANBP1, GEMIN4, MRPS34, DHX33, PPRC1, PPAT, FAM216A/C12ORF24, PAICS, UCHL3, NOLC1, KIAA0226L, PRMT1, LDHB, TRAP1, AHCY, LRP8, EBNA1BP2, CDK4, ETFA, UCK2, CTPS, GOT2, FAM211A/C17ORF76, TMEM97, RRS1, DDX21, PHB2, WDR3, KIAA0101, FASN, SAMD13, CDC25A, LYAR, CAD, APEX1, NOP2, PHB, SSBP1, MRPS2, CIRH1A, SLC16A1, BUB1B, APITD1, NCL, DLEU1, PCDH9, IGFBP2, TDO2, SLC12A8, P2RY12, TMEM119, SHISA8, and SLAMF1; and calculating a MYC activity score based on the mRNA levels; and selecting, excluding or stratifying the subject based on the MYC activity score and/or the diagnostic score. In some embodiments, the methods described herein include determining levels of one or more housekeeping genes, selected from the group consisting of AAMP, H3F3A, HMBS, KARS, PSMB3, and TUBB.

In some embodiments, the methods described herein include normalizing expression levels of the signature genes to the levels of the housekeeping genes.

In some embodiments of the methods described herein, determining a diagnostic score comprises applying a logistic regression model with elastic net regularization to the mRNA levels.

In some embodiments of the methods described herein, determining a MYC activity score applying a logistic regression model with elastic net regularization to the mRNA levels.

In some embodiments of the methods described herein, the mRNA levels are weighted, e.g., using the Gene weights shown in Table 4.

In some embodiments of the methods described herein, the MYC activity score and/or diagnostic score is calculated using a suitably programmed computing device.

In some embodiments of the methods described herein, the MYC activity score and/or diagnostic score is calculated using a logistic regression function. In some embodiments of the methods described herein, the logistic regression function is:

$$p = \frac{1}{1 + {e}^{- ( {β}_{0} + {β}_{1} {x}_{1} + {β}_{2} {x}_{2} + … + {β}_{n} {x}_{n} )}} ,$$

Where p is the probability that a patient belongs to a certain class, i.e., BL in the case of the diagnostic classifier and high MYC in the case of the MYC classifier. β₀ represents the intercept of the logistic regression model, which is 17.4688 for the diagnostic classifier and 32.3287 for the MYC classifier. β_{1 . . . n} are the gene weights as shown in Table 4, and x_{1 . . . n} represent the gene expression values derived from a patient sample.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-B. Target gene selection and the creation of molecular classifiers. (A) Schematic showing the distribution of gene transcripts that were assayed in the initial and final profiling panels. (B) Schematic outlining the protocols for the molecular classification of (i) all aggressive B-cell lymphomas, and (ii) cases with the pathological diagnosis of DLBCL and BCL-U. Twenty-one genes were used for the Diagnostic classifier and 61 genes were used for the MYC Activity classifier. In addition 8 genes were common to both classifiers and there were 6 housekeeping genes.

FIG. 2. Unsupervised clustering of the normalized transcript values from 42 tumors comprising the training cohort and including all target probes in the initial profiling panel (1 case later failed quality control during classification). The original pathological diagnosis (first line) and relative gene expression for the 185 genes comprising the initial profiling panel (heatmap) are shown (15 housekeeping genes excluded).

FIGS. 3A-B. (A) Leave-one-out cross-validation (LOO-CV) of the final profiling panel and Diagnostic Classifier for the training cohort: BL and DLBCL cases categorized according to the original pathological diagnosis (first line), the assigned molecular diagnosis (second line, diagnostic scores of 0.25-0.75 categorized as ‘molecularly intermediate’), diagnostic score (line graph, third line, intermediate values shaded), the relative expression of the indicated transcripts (heatmap) including the relative contribution of each to the classifier (horizontal shaded bar graphs, left side), and MYC-rearrangement status (bottom line). (B) Results of the Diagnostic Classifier for the test cohort: BL, BCL-U and DLBCL cases categorized according to the original pathological diagnosis (first line), the assigned molecular diagnosis (second line, diagnostic scores of 0.25-0.75 categorized as ‘intermediate’), diagnostic score (line graph, third line, intermediate values shaded), the relative expression of the indicated transcripts (heatmap) including the relative contribution of each to the classifier (horizontal shaded bar graphs, left side), and MYC-rearrangement status (bottom line). The cases of genetic DHL are numbered and additional gene rearrangements are indicated by arrowheads (BCL2-) or a dot (BCL6-). The ‘single hit’ DLBCL, with MYC-rearrangement only, is indicated by an asterisk.

FIGS. 4A-B. (A) Scatterplot showing the mean TCF3 signature (7 genes, x axis) and mean MYC signature (10 genes, y axis) for each tumor from the test cohort. The mean values for each signature are derived from transcript counts from these genes, as originally used in the diagnostic classifier. Colors indicate the pathological/genetic diagnoses (black for BL, gray for DLBCL, yellow for genetic DHL). Shapes indicate the molecular classification assigned by the diagnostic classifier (triangle for mBL, circle for mDLBCL, square for molecularly intermediate). (B) Histomorphological features of lymphomas with a MYC-rearrangement and either a BCL2- or BCL6-rearrangement (genetic DHL). Hematoxylin and eosin stained sections of DHL classified as molecular BL (top row), and molecular DLBCL (bottom row). Unique identifiers and details of relevant translocations are shown. Cases were photographed at ×1000 original magnification. The tumors classified as mDLBCL have inserts highlighting nuclear morphology.

FIGS. 5A-B. (A) Leave-one-out cross-validation (LOO-CV) of the final profiling panel and MYC Activity Classifier for the training cohort. BL (left side) and DLBCL (right side) are segregated by pathological diagnosis (first line), MYC activity score (second line and line graph), the relative expression of the indicated transcripts (heatmap) including the relative contribution of each to the classifier (horizontal, shaded bar graphs, left side), MYC IHC class (MYC IHC-Low≦50%, IHC-High>50%; penultimate line) and MYC rearrangement status (bottom line). Inset is the correlation between MYC IHC and MYC activity score for DLBCL only (Spearman r=0.80; 95% CI 0.6-0.9). (B) Results of the final profiling panel and MYC Activity Classifier for the test cohort: BL (left side), DLBCL and BCL-U (right side) are segregated by pathological diagnosis (first line), MYC activity score (second line and line graph), the relative expression of the indicated transcripts (heatmap) including the relative contribution of each to the classifier (horizontal, shaded bar graphs, left side), MYC IHC class (MYC IHC-Low≦50%, IHC-High>50%; penultimate line) and MYC rearrangement status (bottom line). Genetic DHLs are indicated as in FIG. 3(B). Inset is the correlation between MYC IHC and MYC activity score for non-BL only (Spearman r=0.66; 95% CI 0.44-0.8).

FIGS. 6A-B. Results of the MYC classifier and overall survival (OS) among patients with primary DLBCL treated with R-CHOP-based chemotherapy. (A) The correlation between MYC score and MYC IHC for the outcome series (Spearman r=0.64; 95% CI 0.4-0.8). (B) Kaplan-Meier (KM) curve showing Overall Survival (OS) for the outcome series with a MYC score>0.5 (red line) and a MYC score<0.5 (black line).

FIG. 7. Schematic depicting target gene selection. (i) TCF3 genes were derived from Schmitz et al (Nature. 2012 Aug. 12; 490(7418):116-20) and then validated in silico by differential analysis against GEPs of BL and DLBCL from 2 prior publications (Dave et al., New England Journal of Medicine. 2006; 354(23):2431-42; Hummel et al., New England Journal of Medicine. 2006; 354(23):2419-30). Transcripts with “false discovery rates” (FDR)<0.25 were ranked by signal to noise ratio and the top 37 within the union of the two analyses were selected for the initial profiling panel. Analysis of expression data from the training cohort and the construction of molecular classifiers resulted in the inclusion of 7 gene targets in the final probe set that was tested in the test cohort. (ii) MYC biological activity signature was derived from the differential analysis of 457 published MYC targets (Zeller et al., Genome Biol. 2003; 4 (10):R69; Mori et al., Cancer Research. 2008 Oct. 15; 68(20):8525-34; Schuhmacher et al., Nucleic Acids Research. 2001 Jan. 15; 29(2):397-406 Kim et al., Oncogene. 2005 Aug. 22; 25(1):130-8; Chapuy et al., Cancer Cell. 2013; 24(6):777-90; Schlosser et al., Oncogene. 2004 Nov. 1; 24(3):520-4; Yu et al., Annals of the New York Academy of Sciences. 2005; 1059(1):145-59) against the global GEP of frozen DLBCLs with corresponding MYC IHC class (MYC IHC high versus MYC IHC low) from the training cohort. Differential analysis of the entire 18,400 transcripts within the GEP of the frozen tissue with respect to the corresponding MYC IHC class was also performed.

FIG. 8. Unsupervised hierarchical clustering of data for 3 tumors (DLBCL20, DLBCL3, DLBCL10) tested on more than one occasion during the test study. Heatmap data are normalized to the 6 housekeeping genes but are not normalized between ‘profiling panel builds’. The ‘profiling panel build’ and experiment number (first line), the relative expression of the transcripts used in the final profiling panel are shown (heatmap, housekeeping gene data not shown). *The mean MYC activity score (third line) and bar charts of respective MYC activity scores by build and experiment number (fourth line) use the final classifier output, following normalization of data between profiling panel builds.

FIGS. 9A-B. Kaplan-Meier (KM) curves showing Overall Survival (OS) for the outcome series where matched MYC IHC and MYC Activity score data are available. Two cases lacked MYC IHC score therefore n=38 rather than n=40 in FIG. 6. (A) Segregated by MYC IHC: MYC IHC-High>50% (red line) and MYC IHC-Low≦50% (black line). (B) Segregated by MYC Activity Score: MYC Activity High (>0.5, red line) and MYC Activity Low (<0.5, black line).

### DETAILED DESCRIPTION

The World Health Organization (WHO) classification of tumors defines neoplastic diseases according to unique clinical and biological characteristics¹. Burkitt lymphoma (BL) and diffuse large B-cell lymphoma (DLBCL) are aggressive tumors of mature B-cells categorized as individual tumor types. The reliable differentiation of BL from DBLCL is important, as these tumors are treated with distinct chemotherapeutic regimens^{2,3}.

BL is a neoplasm composed of monomorphic, intermediate-sized lymphocytes that are positive for markers of mature, germinal-center B-cells and negative for the anti-apoptotic protein BCL2. The vast majority of cells (>95%) are positive for the proliferation marker Ki67/MIB1. The genetic hallmark of BL is a balanced translocation involving the MYC oncogene and, most commonly, the immunoglobulin heavy chain locus (IGH)^{1,4}. Mutations in TCF3 and ID3 are also common^{5,6}. In contrast, DLBCL is composed of pleomorphic, large lymphoid cells and, in general, less apoptosis and a lower proliferation index than BL. DLBCLs express markers of mature B-cells, with or without evidence of germinal center cell derivation, and a majority express BCL2. Genetically, only a small subset of DLBCLs have a MYC translocation and mutations in TCF3 or ID3 are rare. However, mutations in genes encoding the components of the NF-kB and B-cell receptor signaling pathways are common^{1,7-11}.

Most cases of BL and DLBCL are diagnosed with high confidence using traditional histopathologic, immunophenotypic, and targeted genetic analyses. However, it is not uncommon to encounter tumors with one or more features overlapping BL and DLBCL. The 2008 WHO Classification of Lymphoid Tumors recognized these cases with the novel diagnostic category, “B-cell lymphoma unclassifiable, with features intermediate between DLBCL and BL” (BCL-U)¹. BCL-U is, by definition, a heterogeneous group, and its diagnosis requires that pathologists make subtle distinctions in histomorphology, immunophenotype, and genetics that may not be highly reproducible.

Molecular classification of aggressive B-cell lymphomas using comprehensive gene-expression profiles (GEPs) of RNA isolated from frozen tumor samples accurately differentiates BL from DLBCL and confirms that a subset of cases has transcriptional signatures intermediate between BL and DLBCL^{12,13}. However, the pathological diagnoses corresponding to these ‘biologically intermediate’ tumors have been inconsistent¹³.

Complicating the evaluation of aggressive lymphomas is the recognition that high MYC expression and biological activity, once thought to be only associated with BL, are major, independent predictors of poor clinical outcome among patients with primary DLBCL treated with R-CHOP¹⁴⁻¹⁸. In some series, the prognostic value of MYC is enhanced among tumors that co-express BCL2^{14,19-21}. Indeed recent evidence suggests that high co-expression of MYC and BCL2 in tumor cells provides a biological basis for the inferior outcome among patients with the activated B-cell (ABC) type DLBCL when treated with standard chemotherapy²¹.

DLBCL with high MYC activity cannot be identified with certainty by morphologic or genetic studies alone¹⁵. The detection of MYC in fixed tumor biopsy specimens by immunohistochemistry (IHC) has the potential to identify DLBCLs with high MYC protein that corresponds to high MYC biological activity¹⁵. However, IHC methods are difficult to standardize between institutions and the interpretation of IHC staining is subjective²².

These data highlight a need for quantitative methods that capture the phenotypic, genetic and molecular heterogeneity of aggressive B-cell lymphomas in clinical practice. Molecular classification based upon the unique gene expression profiles of BL, DLBCL, and MYC-driven B-cell lymphomas has the potential to satisfy this need, but, until recently, gene expression profiling (GEP) has not been amenable to FFPE tissues²³⁻²⁶.

Described herein are methods for targeted expression profiling followed by a 2-stage molecular classifier of aggressive mature B-cell lymphomas that is applicable to FFPE biopsy specimens.

Described herein is a framework for the molecular classification of MYC-driven B-cell lymphomas using targeted expression profiling of RNA isolated from FFPE tissue. The approach described has several features that make it appealing. First, the assay requires only small amounts of FFPE tissue. RNA isolated from the equivalent of a 2 to 6×5-μm FFPE tissue sections is sufficient for analysis²⁴. Second, the assay is robust. 96 FFPE tumor biopsy samples ranging from 0.5 to 13 years old were successfully profiled, with only an additional 5 (5%) failing analytical quality control, and repeat testing of the same samples yielded nearly identical results. Third, the step-wise application of the diagnostic and MYC activity classifiers mimics the diagnostic approach used to evaluate aggressive B-cell lymphomas in clinical practice. Finally, the molecular scores provide quantitative outputs that can be interpreted objectively. Thus, the assessment of defined molecular signatures from FFPE tissue, using the methods described here, has the potential to provide important additional biological information alongside traditional diagnostic techniques, to facilitate lymphoma classification.

The definition of BL was framed in terms of high MYC and TCF3 transcriptional activity, as these are known major determinants of tumor behavior⁴⁻⁶. DLBCL was defined by variable MYC activity, low TCF3 activity, and high BCL2 and targets of NFKB¹². This limited signature was sufficient to categorize>90% of BL and DLBCL in the test set with high confidence and with perfect accuracy (Table 5). The results are comparable to those reported in a prior, exploratory study comparing categorization of BL and non-BL using targeted GEP against a ‘gold standard’ global GEP²⁵, and validate a molecular, diagnostic classification for cases of well-defined BL and DLBCL.

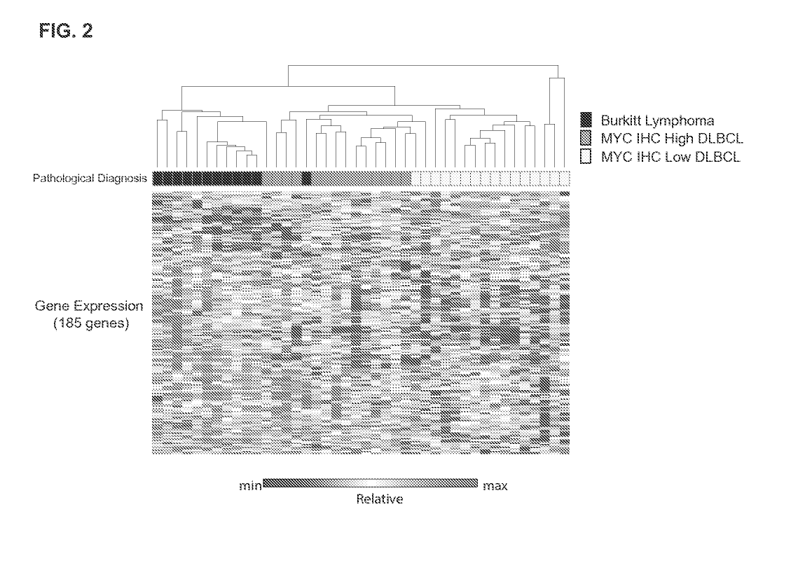

BCL-U are ‘intermediate’ tumors that share features with BL and DLBCL according to traditional diagnostic evaluation, but ‘intermediate’ tumors are also identified by molecular analyses^{1,12,13}. It is important to note that ‘histomorphologically intermediate’ and ‘molecularly intermediate’ are non-synonymous terms and will categorize mature, aggressive B-cell lymphomas in different ways⁴². For example, in our test cohort, 3 BCL-Us classified as mBL. This must be considered inaccurate in the context of WHO classification but is consistent with prior molecular characterization of B-cell lymphomas in which most ‘atypical BLs’ and a proportion of ‘unclassifiable aggressive B-cell lymphomas’ classified as mBL (Hummel et al., FIG. 2 (2006)¹³). Similarly, small numbers of BL, BCL-U, and DLBCL in our series had diagnostic molecular scores ‘intermediate’ between mBL and mDLBCL. This result is also consistent with prior analyses in which subsets of atypical BL, ‘unclassifiable aggressive B-cell lymphoma’, and DLBCL classified as ‘molecularly intermediate’¹³. These results support the concept that BCL-U is not a discrete diagnostic category, but includes tumors with molecular profiles of mBL, mDLBCL, and intermediate between mBL and mDLBCL.

Non-BL with MYC-rearrangement is also a heterogeneous group that includes tumors with the pathological diagnoses of BCL-U and DLBCL by WHO criteria^{1,42-46}. As described herein, there were DHLs that classified as mBL, ‘molecularly intermediate’, and mDLBCL. This result also has precedence. A comprehensive GEP analysis of aggressive B-cell lymphomas highlighted groups of DHLs that classified as mBL and MYC-rearranged DLBCLs that classified as mDLBCL (Dave et al., FIG. 2 (2006)¹²).

The present results were further supported by the examination of the molecular sub-signatures and the histomorphology of the DHLs. A subset of DHLs have a TCF3 signature that is comparable to, or exceeding that of BL. This result was surprising, given recent reports that the TCF3 signature is specific for BL^{5,6}, although a recent study found that ID3 mutations can occur in DHL⁴⁵.

DHLs that classified as mBL were histomorphologically typical of BL and cases that classified as mDLBCL were histomorphologically typical of DLBCL. Morphological heterogeneity among DHLs is recognized and may have clinical significance.

The prognostic role of MYC in DLBCL is well established, especially in the context of BCL2 expression, and an assessment of MYC activity has been proposed to be an important part of the diagnostic work-up^{14-16,19-21,48}. MYC IHC is a single biomarker that serves as a surrogate for MYC activity. The threshold for MYC IHC that separates low from high-risk disease varies between studies from 10-50%, with most suggesting 40%^{14-16,19-21,49}. However, IHC is difficult to standardize between centers, even if an automated platform is used²². Therefore, it was hoped that the MYC activity scores would show good, but not perfect, correlation with MYC IHC scores, which was observed. There are a number of pre-analytical and analytical variables that we must consider when reviewing MYC IHC data, such as time to tissue fixation and intra- and inter-observer variability in assessment. A potential advantage of expression profiling is that the analysis of a large number of gene-transcripts provides redundancy to the assay and captures a transcriptional signature of MYC activity that IHC for MYC alone cannot offer. However, comparing between a single data point (MYC protein expression by IHC) and the combination of a broad set of data (MYC activity score) is also likely to contribute to the observed imperfect correlation between the two methods of assessment. It is also possible that additional MYC targets, not included in our final profiling panel would improve the validity of the MYC activity score.

The MYC activity classifier was trained using the gene expression profiles of DLBCLs alone, excluding BLs. Its subsequent application to BLs in the training and test sets revealed high MYC activity scores for all cases, which supports the validity of the classifier. Moreover, 5 of the 6 non-BLs with the highest MYC activity scores in the test set had MYC-translocations. Yet, tumors with MYC-translocations and intermediate/low scores were also observed; indicating variable MYC activity among SHLs and DHLs^{13,44}.

To evaluate the clinical relevance of these data, the MYC activity scores were correlated to clinical outcome in a small series of R-CHOP-treated patients with primary, de novo DLBCL. Segregating tumors into those with high (>0.5) and low (<0.5) MYC activity scores identified patient populations that differed significantly with respect to overall survival (nominal p=0.0009). The results provide evidence that the MYC activity score, while showing imperfect correlation with IHC and genetics, captures a biological signature of clinical significance. The limited number of primary DLBCLs with documented treatment and outcome required that we include cases from the training and test sets, therefore a more formal validation of the MYC classifier using an independent case series is needed. Ideally, such a study would compare the inter-institutional reproducibility and the prognostic value of the MYC classifier with MYC IHC in a large, multi-institutional cohort.

Thus described herein are quantitative methods for classifying and stratifying aggressive B-cell lymphomas that is applicable to FFPE tissue samples. The molecular classifiers are robust, but might improve with the inclusion of additional, select gene signatures²⁴. In addition to distinguishing BL from DLBCL, the diagnostic classifier provides unique data regarding the further classification of BCL-Us and DHLs that inform the standard diagnostic methods and warrant further investigation. This platform will allow for the standardized analysis of an expanded cohort of BCL-U and DHL, from which correlations between GEP and traditional pathology, genetics, and somatic mutational analysis can be further examined. The MYC activity classifier captures a key biological and prognostic hallmark of DLBCL and also has the potential to standardize assessment across institutions.

Methods of Diagnosis and Prognosis

The methods described herein use a molecular classifier to diagnose subjects with Burkitt lymphoma (BL) and diffuse large B-cell lymphoma (DLBCL), based on analysis of mRNA levels, e.g., RNA from formalin-fixed, paraffin-embedded biopsy specimens. In some embodiments, the methods are used to diagnose BL versus DLBCL in a subject who has B-cell lymphoma with one or more characteristics that overlap BL and DLBCL, e.g., categorized as B-cell lymphoma unclassifiable, with features intermediate between BL and DLBCL (BCL-U) (see above).

The methods include determining mRNA levels of diagnostic signature genes in a cell, and determining a diagnostic score based on the mRNA levels. In some embodiments, the methods include the use of two or more, e.g., all of the diagnostic signature genes as shown in Table 4, i.e., STRBP, PRKAR2B, E2F2, LZTS1, CDC25A, TCF3, RANBP1, DLEU1, PAICS, DNMT3B, PPAT, KIAA0101, PYCR1, CD10, NME1, FAM216A/C12ORF24, BMP7, BCL2, CD44, p50 (NFKB1), and BCL2A. Exemplary reference gene sequences are shown in Table 4.

In addition, the methods can be used to predict outcome in those subjects, based on the use of a molecular classifier of MYC activity levels. For example, these methods can be used to predict response to treatment, e.g., treatment with the R-CHOP (rituximab, cyclophosphamide, doxorubicin, vincristine, and prednisone) regimen. The methods can also be used to predict survival.

The methods include determining mRNA levels of MYC activity signature genes in a cell, and determining a MYC activity score based on the mRNA levels. In some embodiments, the methods include the use of two or more, e.g., all of the MYC activity signature genes as shown in Table 4, i.e., MYC, SRM, AKAP1, NME1, FBL, RFC3, TCL1A, POLD2, RANBP1, GEMIN4, MRPS34, DHX33, PPRC1, PPAT, FAM216A/C12ORF24, PAICS, UCHL3, NOLC1, KIAA0226L, PRMT1, LDHB, TRAP1, AHCY, LRP8, EBNA1BP2, CDK4, ETFA, UCK2, CTPS, GOT2, FAM211A/C17ORF76, TMEM97, RRS1, DDX21, PHB2, WDR3, KIAA0101, FASN, SAMD13, CDC25A, LYAR, CAD, APEX1, NOP2, PHB, SSBP1, MRPS2, CIRH1A, SLC16A1, BUB1B, APITD1, NCL, DLEU1, PCDH9, IGFBP2, TDO2, SLC12A8, P2RY12, TMEM119, SHISA8, and SLAMF1. Exemplary reference gene sequences are shown in Table 4.

In some embodiments, the methods include determining both a diagnostic score and a MYC activity score, as described above, based on expression levels of all of the signature genes listed in Table 4. In some embodiments, only levels of mRNA for genes that appear in both the diagnostic and MYC activity sets, e.g., the 8 genes in bold in Table 4, are determined, and diagnostic and MYC activity scores determined based thereon.

In some embodiments, the methods include determining levels of one or more housekeeping genes, e.g., selected from the group consisting of AAMP, H3F3A, HMBS, KARS, PSMB3, and TUBB. The methods can include normalizing expression levels of the signature genes to the levels of the housekeeping genes. Exemplary reference gene sequences are shown in Table 4.

Although the exemplary gene sequences set forth herein are for the human genes, and thus are best suited for use in human cells, one of skill in the art could readily identify mammalian homologs using database searches (for known sequences) or routine molecular biological techniques (to identify additional sequences). In general, genes are considered homologs if they show at least 80%, e.g., 90%, 95%, or more, identity in conserved regions (e.g., biologically important regions).

In some embodiments, a subject having a B-cell lymphoma, e.g., a BCL-U, is identified (methods for diagnosing the presence of BCL are well known in the art and need not be repeated herein). A test sample is obtained from the tumor, e.g., a Formalin-Fixed, Paraffin-Embedded sample (FFPE). Although FFPE samples are exemplified, others can be used, e.g., fresh frozen tissue sections, fine needle aspirate biopsies, tissue microarrays, cells isolated from blood (including whole blood), bone marrow or sputum (such as samples prepared using centrifugation (such as with the CytoSpin Cytocentrifuge instrument (ThermoFisher Scientific, Waltham, Mass.) or smeared on a slide), blood smears on slides (including whole blood smears), and other sample types.

Any method known in the art can be used to extract material, e.g., nucleic acid (e.g., mRNA) from the sample. For example, mechanical or enzymatic cell disruption can be used, followed by a solid phase method (e.g., using a column) or phenol-chloroform extraction, e.g., guanidinium thiocyanate-phenol-chloroform extraction of the RNA. A number of kits are commercially available for use in isolation of mRNA. Purification can also be used if desired. See, e.g., Peirson and Butler, Methods Mol. Biol. 2007; 362:315-27. Optionally, cDNA can be transcribed from the mRNA.

The levels of signature (and optional housekeeping) mRNAs is evaluated using methods known in the art, e.g., using multiplexed gene expression analysis methods, e.g., RT-PCR, RNA-sequencing, and oligo hybridization assays including RNA expression microarrays, hybridization based digital barcode quantification assays such as the nCounter® System (NanoString Technologies, Inc., Seattle, Wash.; Kulkarni, Curr Protoc Mol Biol. 2011 April; Chapter 25:Unit25B.10), and lysate based hybridization assays utilizing branched DNA signal amplification such as the QuantiGene 2.0 Single Plex and Multiplex Assays (Affymetrix, Inc., Santa Clara, Calif.; see, e.g., Linton et al., J Mol Diagn. 2012 May-June; 14(3):223-32); SAGE, high-throughput sequencing, multiplex PCR, MLPA, luminex/XMAP, or branched DNA analysis methods. As used herein, a “test sample” refers to a biological sample obtained from a subject of interest including a cell or cells, e.g., tissue, from the tumor.

In some embodiments the methods include contacting the sample with a detectably labeled probe or probes. Thus in some embodiments, the methods include the use of alkaline phosphatase conjugated polynucleotide probes. Where an alkaline phosphatase (AP)-conjugated polynucleotide probe is used, following sequential addition of an appropriate substrate such as fast red or fast blue substrate, AP breaks down the substrate to form a precipitate that allows in-situ detection of the specific target RNA molecule. Alkaline phosphatase can be used with a number of substrates, e.g., fast red, fast blue, or 5-Bromo-4-chloro-3-indolyl-phosphate (BCIP). See, e.g., as described generally in U.S. Pat. No. 5,780,277 and U.S. Pat. No. 7,033,758.

Other embodiments include the use of fluorophore-conjugates probes, e.g., Alexa Fluor conjugated label probes, or utilize other enzymatic approaches besides alkaline phosphatase for a chromogenic detection route, such as the use of horseradish peroxidase conjugated probes with substrates like 3,3′-Diaminobenzidine (DAB).

In some embodiments, the methods include applying an algorithm to expression level data determined in a cell; e.g., an elastic net model as described herein. In some embodiments, the algorithm includes weighting coefficients for each of the genes, e.g., the “variable importance” weights as shown in Table 4. Alternatively, linear or polynomial support vector machines, shrunken centroids, or a random forest algorithm can be used in place of the elastic net prediction model.

In some embodiments, the methods include applying an algorithm to expression level data determined in a cell; e.g., a logistic regression model using elastic net regularization (elastic net) as described herein. In some embodiments, the algorithm includes weighting coefficients for each of the genes, e.g., the gene weights as shown in Table 4. Alternatively, linear or polynomial support vector machines, shrunken centroids, or a random forest algorithm can be used in place of the elastic net prediction model.

A logistic regression model useful in the methods described herein includes gene expression levels and coefficients, or weights, for combining expression levels.

A logistic regression model using elastic net regularization as used in some embodiments of the methods described herein is a statistical machine learning method that is capable of selecting, weighting and combining single gene expression values. The elastic net regularization is used to select the most relevant genes that make up the classification model. The actual classifier, the logistic regression is then used to map the single gene expression values from a multi-dimensional space onto a scale between 0 and 1, which resemble probability values or activation scores. The logistic regression function looks as follows:

$$p = \frac{1}{1 + {e}^{- ( {β}_{0} + {β}_{1} {x}_{1} + {β}_{2} {x}_{2} + … + {β}_{n} {x}_{n} )}}$$

Where p is the probability that a patient belongs to a certain class, i.e., BL in the case of the diagnostic classifier and high MYC in the case of the MYC classifier. β₀ represents the intercept of the logistic regression model, which is 17.4688 for the diagnostic classifier and 32.3287 for the MYC classifier. β_{1 . . . n} are the gene weights as shown in Table 4 and x_{1 . . . n} represent the gene expression values derived from a patient sample.

In some embodiments, an elastic net regularization with an a parameter of 0.1 and a λ parameter of 0.1 was used to build a logistic regression classification model for both diagnosis and prognosis. In some embodiments, a diagnostic score of >0.75 represents molecular BL (mBL), and <0.25 represents molecular DLBCL (mDLBCL), and 0.25 to 0.75 represents molecularly intermediate. In addition, as noted herein, MYC activity scores of 1 and 0 corresponded to tumors with high MYC and low MYC (as modeled on IHC expression) with greatest probability, respectively, and a MYC activity score of 0.5 as the cutoff with the highest estimated accuracy to classify tumors with high and low MYC activity. Therefore, in some embodiments a MYC activity score of 0.5 is used as the cutoff to classify tumors with high and low MYC activity and for correlation to clinical outcome.

Methods of Selecting Treatment

The methods described herein can also be used to select treatment for a subject. For example, tumors with high levels of MYC activity respond poorly to traditional chemotherapy, e.g., to the R-CHOP regimen. Thus, the methods can include determining a level of MYC activity using the MYC activity signature and selecting (and optionally administering) standard chemotherapy for subjects with a low MYC activity score, or selecting (and optionally administering) a different treatment for subjects with a high MYC activity score. In some embodiments a MYC activity score of 0.5 is used as the cutoff to classify tumors with high or low MYC activity.

In addition, the methods can include selecting a treatment based on the diagnostic score; for example, in some embodiments, a diagnostic score below a first threshold (e.g., of <0.25) represents molecular DLBCL (mDLBCL), and the subject should be administered a treatment for DLBCL. For example, for Stage I/II (nonbulky) disease, the treatment could include Rituximab (R) plus cyclophosphamide, vincristine, doxorubicin, and prednisone (CHOP) for 3-4 cycles (R-CHOP), optionally followed with involved field radiation therapy (IFRT). If positron emission tomography (PET) is positive after the first 4 cycles, 2 more cycles of R-CHOP can be administered before IFRT.

For advanced-stage (stage III-IV) or bulky stage II disease, the treatment can include R+CHOP every 21 d for 6 cycles, with or without IFRT for bulky sites. Prophylactic intrathecal (IT) chemotherapy or inclusion in a clinical trial with correlative science studies (eg, R+CHOP-like and other biological agents or small molecules and/or other novel monoclonal antibodies [mAbs] or immunoconjugates) can also be used.

For relapses, eligible patients can receive treatment with high-dose chemotherapy (HDC) and autologous stem cell transplantation (ASCT), e.g., platinum-based salvage chemotherapy, including rituximab, ifosfamide, carboplatin, and etoposide (RICE) for 2-3 cycles, or rituximab plus cisplatin, cytarabine, and dexamethasone (DHAP) for 2-3 cycles. If partial or complete response is achieved, HDC and ASCT can be used together. Other agents can also be used (e.g., bortezomib, lenalidomide, or immunoconjugates) or radioimmunotherapy (RIT). See, e.g., Hernandez-Ilizaliturri, Diffuse Large B-Cell Lymphoma (Non-Hodgkin Lymphoma) Treatment Protocols, emedicine.medscape.com/article/2005945-overview (May 15, 2013).

In other embodiments, a diagnostic score above a second threshold (e.g., of >0.75) represents molecular BL (mBL), and the subject should be administered an aggressive treatment for BL, e.g., intensive systemic chemotherapy such as intensive, short-duration regimens like CODOX-M/IVAC (Magrath regimen) and the CALGB 9251 protocol; long-duration chemotherapy similar to acute lymphoblastic leukemia (ALL) treatment, like hyper-CVAD and the CALGB 8811 protocol; combination regimens followed by autologous stem cell transplantation (SCT). Rituximab can be added as well.

CODOX-M is Cyclophosphamide 800 mg/m 2 IV on day 1, followed by 200 mg/m 2 IV on days 2-5; Doxorubicin 40 mg/m 2 IV on day 1; Vincristine 1.5 mg/m 2 IV (no capping of dose) on days 1 and 8 (cycle 1), as well as on days 1, 8, and 15 (cycle 3); Methotrexate 1200 mg/m 2 IV over 1 hour on day 10; then 240 mg/m 2/h for the next 23 hours; leucovorin rescue begins 36 hours from the start of the methotrexate infusion; Intrathecal cytarabine 70 mg (patient older than age 3 y) on days 1 and 3; Intrathecal methotrexate 12 mg (patient older than age 3 y) on day 15.

The CODOX-M/IVAC (cyclophosphamide, vincristine, doxorubicin, high-dose methotrexate/ifosfamide, etoposide, high-dose cytarabine) regimen consists of 4 cycles, each cycle lasting until blood counts recover (absolute neutrophil count [ANC]>1000/μL; platelets>100,000/μL). Cycles 1 and 3 involve CODOX-M, and cycles 2 and 4 involve IVAC. Three cycles of CODOX-M are usually enough for low-risk patients, whereas high-risk patients receive 4 total cycles (2 cycles of CODOX-M, alternating with 2 cycles of IVAC).

The IVAC protocol includes Ifosfamide 1500 mg/m² IV on days 1-5, with mesna protection; Etoposide 60 mg/m² IV on days 1-5; Cytarabine 2 g/m² IV every 12 hours on days 1-2; Intrathecal methotrexate 12 mg (patient older than age 3 y) on day 5; and administration of colony-stimulating factors, usually initiated 24 hours after completion of chemotherapy and continues until the ANC>1000/μL.

Other treatment regimens for BL include the CALGB Regimen, e.g., as described in Lee et al., J Clin Oncol. Oct. 15 2001; 19(20):4014-22 and Rizzieri et al., Cancer. Apr. 1 2004; 100(7):1438-48, and the hyper-CVAD (modified fractionated cyclophosphamide, vincristine, doxorubicin, and dexamethasone) regimen. See, e.g., Kandar and Sacher, Burkitt Lymphoma and Burkitt-like Lymphoma Treatment & Management, available at emedicine.medscape.com/article/1447602-treatment (Mar. 31, 2014); see also, McMaster et al., J Clin Oncol. June 1991; 9(6):941-6. Rituximab can be added to any of the above (e.g., R-Hyper-CVAD, R-CODOX-M/IVAC).

In some embodiments, the second threshold is higher than the first threshold, and a score between the first and second thresholds (e.g., 0.25 to 0.75) represents molecularly intermediate. In other embodiments, the first and second thresholds are the same number.

Traditional chemotherapy, to be selected for (and optionally administered to) those with low MYC activity, can include, e.g., the R-CHOP regimen (see, e.g., refs. 14-18), as well as those treatments listed above for DLBCL.

Alternative chemotherapy, to be selected for (and optionally administered to) those with high MYC activity, can include those treatments listed above for BL (e.g., R-Hyper-CVAD, R-CODOX-M/IVAC), as well as DA-EPOCH-R (dose-adjusted etoposide, prednisone, vincristine, cyclophosphamide, doxorubicin and rituximab); as well as PI3K inhibitors; small-molecule inhibitors of the bromodomain and extraterminal (BET) domain proteins, e.g., JQ1 and I-BET 151; aurora kinase inhibitors, e.g., alisertib; BCL2 inhibitors, e.g., Navitoclax and ABT-199; and BCL6 inhibitors. See, e.g., Dunleavy, Hematology Am Soc Hematol Educ Program. 2014 Dec. 5; 2014(1):107-12, and references cited therein.

Methods of Stratifying Subjects in a Clinical Trial

The MYC activity scores and diagnostic scores can also be used to design cohorts for clinical trials. For example, the methods can include determining a score as described herein, and selecting a subject for inclusion in or exclusion from a clinical trial, or for assignment to a particular cohort in a trial. These methods are particularly useful for selecting and stratifying subjects for treatments that are intended to improve the outcome in subjects with high MYC activity cancers (e.g., DHL).

Kits

The invention also includes kits for detecting and quantifying the selected signature genes (e.g., mRNA) in a biological sample. For example, the kit can include a compound or agent capable of detecting mRNA corresponding to the signature genes in a biological sample; and a standard; and optionally one or more reagents necessary for performing detection, quantification, or amplification. The compounds, agents, and/or reagents can be packaged in a suitable container. The kit can further comprise instructions for using the kit to detect and quantify signature protein or nucleic acid.

For example, the kit can include: (1) an oligonucleotide, e.g., a detectably labeled oligonucleotide, which hybridizes to a nucleic acid sequence corresponding to a signature gene or (2) a pair of primers useful for amplifying a nucleic acid molecule corresponding to a signature gene. The kit can also include a buffering agent, a preservative, and/or a protein stabilizing agent. The kit can also include components necessary for detecting the detectable agent (e.g., an enzyme or a substrate). The kit can also contain a control sample or a series of control samples which can be assayed and compared to the test sample contained. Each component of the kit can be enclosed within an individual container and all of the various containers can be within a single package, along with instructions for interpreting the results of the assays performed using the kit.

In some embodiments, the kits include reagents specific for the quantification of the signature genes listed in a profile shown in Table 4. In some embodiments, the kits also include primers or antibodies selective for a housekeeping or control gene, e.g., as listed in table 4.

Computer Software/Hardware

Standard computing devices and systems can be used and implemented, e.g., suitably programmed, to perform the methods described herein, e.g., to perform the calculations needed to determine the scores described herein. Computing devices include various forms of digital computers, such as laptops, desktops, mobile devices, workstations, personal digital assistants, servers, blade servers, mainframes, and other appropriate computers. In some embodiments, the computing device is a mobile device, such as personal digital assistant, cellular telephone, smartphone, tablet, or other similar computing device. The components described herein, their connections and relationships, and their functions, are meant to be exemplary only, and are not meant to limit implementations of the inventions described and/or claimed in this document.

Computing devices typically include one or more of a processor, memory, a storage device, a high-speed interface connecting to memory and high-speed expansion ports, and a low speed interface connecting to low speed bus and storage device. Each of the components are interconnected using various busses, and can be mounted on a common motherboard or in other manners as appropriate. The processor can process instructions for execution within the computing device, including instructions stored in the memory or on the storage device to display graphical information for a GUI on an external input/output device, such as a display coupled to a high speed interface. In other implementations, multiple processors and/or multiple buses can be used, as appropriate, along with multiple memories and types of memory. Also, multiple computing devices can be connected, with each device providing portions of the operations (e.g., as a server bank, a group of blade servers, or a multi-processor system).

The memory stores information within the computing device. In some embodiments, the memory is a computer-readable medium. In one implementation, the memory is a volatile memory unit or units. In another implementation, the memory is a non-volatile memory unit or units.

The storage device is capable of providing mass storage for the computing device. In one implementation, the storage device is a computer-readable medium. In various different implementations, the storage device can be a floppy disk device, a hard disk device, an optical disk device, or a tape device, a flash memory or other similar solid state memory device, or an array of devices, including devices in a storage area network or other configurations. In one implementation, a computer program product is tangibly embodied in an information carrier. The computer program product contains instructions that, when executed, perform one or more methods, such as those described above. The information carrier is a computer- or machine-readable medium, such as the memory, the storage device, memory on processor, or a propagated signal.

The high speed controller manages bandwidth-intensive operations for the computing device, while the low speed controller manages lower bandwidth-intensive operations. Such allocation of duties is exemplary only. In one implementation, the high-speed controller is coupled to memory, the display (e.g., through a graphics processor or accelerator), and to high-speed expansion ports, which can accept various expansion cards (not shown). In the implementation, the low-speed controller is coupled to a storage device and low-speed expansion port. The low-speed expansion port, which can include various communication ports (e.g., USB, Bluetooth, Ethernet, wireless Ethernet) can be coupled to one or more input/output devices, such as a keyboard, a pointing device, a scanner, or a networking device such as a switch or router, e.g., through a network adapter.

The computing device can be implemented in a number of different forms. For example, it can be implemented as a standard server, or multiple times in a group of such servers. It can also be implemented as part of a rack server system. In addition, it can be implemented in a personal computer such as a laptop computer. Alternatively, components from the computing device can be combined with other components in a mobile device. Each of such devices can contain one or more computing devices, and an entire system can be made up of multiple computing devices communicating with each other.

The computing device typically includes a processor, memory, an input/output device such as a display, a communication interface, and a transceiver, among other components. The device can also be provided with a storage device, such as a microdrive or other device, to provide additional storage. Each of these components are interconnected using various buses, and several of the components can be mounted on a common motherboard or in other manners as appropriate.

The processor can process instructions for execution within the computing device, including instructions stored in the memory. The processor can also include separate analog and digital processors. The processor can provide, for example, for coordination of the other components of the device, such as control of user interfaces, applications run by the device, and wireless communication by the device.

The processor can communicate with a user through control interface and display interface coupled to a display. The display can be, for example, a TFT LCD display or an OLED display, or other appropriate display technology. The display interface can comprise appropriate circuitry for driving the display to present graphical and other information to a user. The control interface can receive commands from a user and convert them for submission to the processor. In addition, an external interface can be provide in communication with the processor, so as to enable near area communication of device with other devices. External interface can provide, for example, for wired communication (e.g., via a docking procedure) or for wireless communication (e.g., via Bluetooth or other such technologies).

The memory stores information within the computing device. In one implementation, the memory is a computer-readable medium. In one implementation, the memory is a volatile memory unit or units. In another implementation, the memory is a non-volatile memory unit or units. Expansion memory can also be provided and connected to the device through an expansion interface, which can include, for example, a SIMM card interface. Such expansion memory can provide extra storage space for device, or can also store applications or other information for the device. Specifically, expansion memory can include instructions to carry out or supplement the processes described above, and can include secure information also. Thus, for example, expansion memory can be provided as a security module for the device, and can be programmed with instructions that permit secure use of the device. In addition, secure applications can be provided via the SIMM cards, along with additional information, such as placing identifying information on the SIMM card in a non-hackable manner.

The memory can include for example, flash memory and/or MRAM memory, as discussed below. In one implementation, a computer program product is tangibly embodied in an information carrier. The computer program product contains instructions that, when executed, perform one or more methods, such as those described above. The information carrier is a computer- or machine-readable medium, such as memory, expansion memory, memory on processor, or a propagated signal.

The device can communicate wirelessly through a communication interface, which can include digital signal processing circuitry where necessary. The communication interface can provide for communications under various modes or protocols, such as GSM voice calls, SMS, EMS, or MMS messaging, CDMA, TDMA, PDC, WCDMA, CDMA2000, or GPRS, among others. Such communication can occur, for example, through a radio-frequency transceiver. In addition, short-range communication can occur, such as using a Bluetooth, WiFi, or other such transceiver.

The device can also communication audibly using audio codec, which can receive spoken information from a user and convert it to usable digital information. Audio codex can likewise generate audible sound for a user, such as through a speaker, e.g., in a handset of device. Such sound can include sound from voice telephone calls, can include recorded sound (e.g., voice messages, music files, etc.) and can also include sound generated by applications operating on device.

The computing device can be implemented in a number of different forms, as shown in the figure. For example, it can be implemented as a cellular telephone. It can also be implemented as part of a smartphone, tablet, personal digital assistant, or other similar mobile device.

Where appropriate, the systems and the functional operations described in this specification can be implemented in digital electronic circuitry, or in computer software, firmware, or hardware, including the structural means disclosed in this specification and structural equivalents thereof, or in combinations of them. The techniques can be implemented as one or more computer program products, i.e., one or more computer programs tangibly embodied in an information carrier, e.g., in a machine readable storage device or in a propagated signal, for execution by, or to control the operation of, data processing apparatus, e.g., a programmable processor, a computer, or multiple computers. A computer program (also known as a program, software, software application, or code) can be written in any form of programming language, including compiled or interpreted languages, and it can be deployed in any form, including as a standalone program or as a module, component, subroutine, or other unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub programs, or portions of code). A computer program can be deployed to be executed on one computer or on multiple computers at one site or distributed across multiple sites and interconnected by a communication network.

The processes and logic flows described in this specification can be performed by one or more programmable processors executing one or more computer programs to perform the described functions by operating on input data and generating output. The processes and logic flows can also be performed by, and apparatus can be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, the processor will receive instructions and data from a read only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. Information carriers suitable for embodying computer program instructions and data include all forms of non volatile memory, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, aspects of the described techniques can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The techniques can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation, or any combination of such back-end, middleware, or front-end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

One computer-implemented modeling algorithm is described herein (namely, the elastic net analysis), although such algorithms themselves are generally outside the scope of the present invention. Other software-based modeling algorithms can also be utilized, alone or in combination, such as the classification or decision trees, linear and polynomial support vector machines (SMV), shrunken centroids, random forest algorithm, support vector machines or neural networks.

### EXAMPLES

The invention is further described in the following examples, which do not limit the scope of the invention described in the claims.

Materials and Methods

The following materials and methods were used in the Examples set forth herein.

Tumor and Patient Cohorts

This study was performed with approval from the institutional review boards of Brigham and Women's Hospital (BWH) and Massachusetts General Hospital (MGH). For each case, one or both of the corresponding pathologists of this study (SJR and AS) reviewed hematoxylin and eosin (H&E) stained slides and the original diagnostic reports to ensure that the final diagnosis fulfilled 2008 WHO diagnostic criteria.

The training set (n=41) comprises 12 BLs and 29 DLBCLs (one additional DLBCL later failed analytical quality control). The BLs were selected based on the quality of available tissue and include all BL subtypes, as well as pediatric and adult patients (median age of diagnosis 30.5 years, range 3-62 years, Table 1). The DLBCLs were selected from a previously published larger series of adult patients¹⁵ who had all been diagnosed as ‘DLBCL-NOS’ (DLBCL not otherwise specified). Previously, MYC IHC-High was defined as >50% expression in tumor cells, and MYC IHC-Low was defined as <50%¹⁵. For training, cases were deliberately selected in order to represent the ‘extremes’ of MYC IHC-High (median 70%; n=13) and MYC IHC-Low (median 20-30%; n=16) in order to assist development of the MYC activity classifier. DLBCLs were not selected with regard to ‘cell of origin’ (COO²⁷) subtype, but previously classified using GEP as 10 ABC-type (34.5%), 13 GCB-type (44.8%), 5 ‘Type 3’ (17.2%) and 1 (3.4%) unclassified (Table 1).

The test set (n=55) is composed of 9 BLs (all adult patients, 8 sporadic and 1 immunodeficiency-associated), 41 DLBCLs and 5 BCL-Us (Table 1). Four additional cases failed analytical quality control. Eight of these tumors are ‘genetic double hit lymphomas’ (DHLs), for the purposes of this study defined as the combination of a MYC-rearrangement and either a BCL2- or BCL6-rearrangement, and these are divided into 3 tumors with a pathological diagnosis of DLBCL and 5 tumors with a pathological diagnosis of BCL-U. The DLBCLs include these 3 DHLs, which are characterized by a combination of MYC and BCL2-rearrangements, as well as 1 ‘single hit lymphoma’ (SHL), characterized by a MYC-rearrangement in isolation. The DLBCLs were chosen on the basis of the quality of available biopsy material and in order to represent a full range of MYC IHC expression. DLBCLs for the test set were not selected on the basis of ‘cell of origin’ (COO²⁷) subtype. COO classification data, using GEP (if available) and/or Han's IHC criteria^{28,29}, showed a distribution of 16 ABC/non-GCB type (39%), 17 GCB-type (41.5%), 3 ‘Type 3’ (7.3%) and 5 (12.2%) unclassified (Table 1). The 5 BCL-Us were selected on the basis of available cases and were all DHLs. Four of the 5 BCL-Us were characterized by a combination of MYC and BCL2-rearrangements and the remaining case had concurrent MYC and BCL6-rearrangements.

Patients included in an outcome cohort (‘Outcome series’; n=40, 22 patients from the training set and 18 from the test set) were derived from a single institution (BWH). All had confirmed primary DLBCL and received standard immuno-chemotherapy (R-CHOP: rituximab, cyclophosphamide, doxorubicin, vincristine, prednisone) as previously reported¹⁵. All clinical data were collected prior to, and independent of, the reference and index tests reported in this study.

TABLE 1
Clinical details of the training and test cohorts are shown. Tumors with no
recorded values for the diagnostic and MYC activity classifiers failed analytical quality
control and were excluded from the training and test sets.
Training Cohort: Burkitt Lymphoma

| | | | | Age of | | | Age of | | MYC | MYC | |
|---|---|---|---|---|---|---|---|---|---|---|---|
| | Figure | | | patient | | | Biopsy | Diagnostic | Activity | IHC | MYC |
| Case | Code | Diagnosis | Subtype | (years) | Sex | Site of Biopsy | (years) | Score | Score | (%) | Rearrangement* |
| 1 | BL1 | Burkitt | Endemic | 3 | M | Submandibular | 6 | 0.999 | 0.945 | 80 | — |
| | | Lymphoma | | | | mass | | | | | |
| 2 | BL2 | Burkitt | Sporadic | 25 | F | Right Adnexal | 12 | 0.996 | 1 | 60 | 1 |
| | | Lymphoma | | | | Mass | | | | | |
| 3 | BL3 | Burkitt | Sporadic | 8 | M | Neck mass | 6 | 0.996 | 0.998 | — | — |
| | | Lymphoma | | | | | | | | | |
| 4 | BL4 | Burkitt | Endemic | 9 | M | Tibial Mass | 6 | 0.996 | 0.994 | 60 | — |
| | | Lymphoma | | | | | | | | | |
| 5 | BL5 | Burkitt | Sporadic | 32 | F | Epidural Mass | 9 | 0.893 | 0.982 | 70 | 1 |
| | | Lymphoma | | | | | | | | | |
| 6 | BL6 | Burkitt | Sporadic | 29 | M | Lymph Node | 9 | 0.85 | 0.996 | 80 | 1 |
| | | Lymphoma | | | | | | | | | |
| 7 | BL7 | Burkitt | Sporadic | 21 | M | Mediastinal Mass | 5 | 0.845 | 0.997 | 100 | 1 |
| | | Lymphoma | | | | | | | | | |
| 8 | BL8 | Burkitt | Immunodeficiency | 62 | M | Parotid mass | 4 | 0.841 | 0.994 | 100 | 1 |
| | | Lymphoma | | | | | | | | | |
| 9 | BL9 | Burkitt | Immunodeficiency | 41 | M | Lymph Node | 9 | 0.829 | 1 | 100 | 1 |
| | | Lymphoma | | | | | | | | | |
| 10 | BL10 | Burkitt | Sporadic | 32 | F | Lymph Node | 9 | 0.632 | 0.629 | 80 | 1 |
| | | Lymphoma | | | | | | | | | |
| 11 | BL11 | Burkitt | Immunodeficiency | 47 | M | Lymph Node | 6 | 0.551 | 0.999 | 100 | 1 |
| | | Lymphoma | | | | | | | | | |
| 12 | BL12 | Burkitt | Sporadic | 41 | F | Retroperitoneal | 6 | 0.306 | 0.952 | 70 | 1 |
| | | Lymphoma | | | | Mass | | | | | |

Training Cohort: DLBCL

| | | | Age of | | | Age of | | MYC | MYC | BCL2 | | | | |
|---|---|---|---|---|---|---|---|---|---|---|---|---|---|---|
| | Figure | | Patient | | Site of | Biopsy | Diagnostic | Activity | IHC | IHC | MYC- | CD5 | EBV | |
| Case | Code | Diagnosis | (years) | Sex | Biopsy | (years) | Score | Score | (%) | (%) | R* | IHC | (EBERs) | COO^{¶} |
| 1 | DLBCL1 | DLBCL- | 64 | F | Retroperit. | 7 | 0.527 | 0.91 | 80 | 10 | 1 | 0 | 0 | GCB |
| | | NOS | | | Mass | | | | | | | | | |
| 2 | DLBCL2 | DLBCL- | 57 | M | Soft tissue | 9 | 0.348 | 1 | 90 | 100 | 1 | 0 | 0 | GCB* |
| | | NOS | | | mass | | | | | | | | | |
| 3 | DLBCL3 | DLBCL- | 49 | F | Lymph node | 5 | 0.305 | 1 | 90 | 60 | 1 | 0 | 0 | GCB |
| | | NOS | | | | | | | | | | | | |
| 4 | DLBCL4 | DLBCL- | 83 | M | Spleen | 5 | 0.207 | 0.58 | 70 | 0 | 1 | 0 | 0 | GCB |
| | | NOS | | | | | | | | | | | | |
| 5 | DLBCL5 | DLBCL- | 49 | M | Lymph node | 9 | 0.164 | 0.84 | 70 | 0 | 0 | 0 | 0 | GCB |
| | | NOS | | | | | | | | | | | | |
| 6 | DLBCL6 | DLBCL- | 70 | F | Lymph node | 7 | 0.122 | 0.76 | 60 | 100 | 0 | 0 | 0 | ABC |
| | | NOS | | | | | | | | | | | | |
| 7 | DLBCL7 | DLBCL- | 66 | F | Lymph node | 6 | 0.113 | 0.49 | 70 | 0 | 0 | 0 | 0 | Type 3 |
| | | NOS | | | | | | | | | | | | |
| 8 | DLBCL8 | DLBCL- | 65 | F | Lymph node | 7 | 0.107 | 0.84 | 70 | 10 | 1 | 0 | 0 | ABC |
| | | NOS | | | | | | | | | | | | |
| 9 | DLBCL9 | DLBCL- | — | — | Unknown | 8 | 0.08 | 0.22 | 30 | 0 | 0 | 0 | 0 | GCB |
| | | NOS- | | | | | | | | | | | | |
| 10 | DLBCL10 | DLBCL- | 63 | F | Supraorb. | 8 | 0.08 | 0.06 | 30 | 0 | 0 | 0 | 0 | GCB |
| | | NOS | | | mass | | | | | | | | | |
| 11 | DLBCL11 | DLBCL- | 75 | M | Lymph node | 5 | 0.065 | 0.98 | 70 | 100 | 0 | 0 | 0 | GCB* |
| | | NOS | | | | | | | | | | | | |
| 12 | DLBCL12 | DLBCL- | 68 | F | Lymph node | 7 | 0.05 | 0.02 | 10 | 100 | 0 | 0 | 0 | GCB* |
| | | NOS | | | | | | | | | | | | |
| 13 | DLBCL13 | DLBCL- | 46 | M | Lymph node | 7 | 0.046 | 0.42 | 20 | 10 | 0 | 0 | 0 | Type 3 |
| | | NOS | | | | | | | | | | | | |
| 14 | DLBCL14 | DLBCL- | 72 | M | Lymph node | 7 | 0.027 | 0.46 | 70 | 100 | 0 | 0 | 0 | ABC |
| | | NOS | | | | | | | | | | | | |
| 15 | DLBCL15 | DLBCL- | 40 | M | Nasoph. | 6 | 0.022 | 0.46 | 40 | 100 | 0 | 0 | 0 | ABC |
| | | NOS | | | mass | | | | | | | | | |
| 16 | DLBCL16 | DLBCL- | 81 | F | Lymph node | 7 | 0.021 | 0.88 | 60 | 70 | 1 | 1 | 1 | ABC |
| | | NOS | | | | | | | | | | | | |
| 17 | DLBCL17 | DLBCL- | — | — | Unknown | 9 | 0.019 | 0.12 | 30 | 40 | 0 | 0 | 0 | ABC |
| | | NOS | | | | | | | | | | | | |
| 18 | DLBCL18 | DLBCL- | 69 | M | Perisplenic | 5 | 0.018 | 0.19 | 10 | 90 | 0 | 0 | 0 | Type 3 |
| | | NOS | | | mass | | | | | | | | | |
| 19 | DLBCL19 | DLBCL- | 60 | M | Lymph node | 6 | 0.016 | 0.01 | 20 | 70 | 0 | 0 | 0 | Type 3 |
| | | NOS | | | | | | | | | | | | |
| 20 | DLBCL20 | DLBCL- | 76 | M | Small bowel | 8 | 0.014 | 0.02 | 20 | 100 | 0 | 0 | 0 | GCB |
| | | NOS | | | | | | | | | | | | |
| 21 | DLBCL21 | DLBCL- | 75 | M | Lymph node | 9 | 0.013 | 0.98 | 30 | 100 | 0 | 0 | 0 | GCB |
| | | NOS | | | | | | | | | | | | |
| 22 | DLBCL22 | DLBCL- | 62 | M | Lymph node | 8 | 0.013 | 0.95 | 80 | 90 | 1 | 0 | 0 | ABC |
| | | NOS | | | | | | | | | | | | |
| 23 | DLBCL23 | DLBCL- | 45 | F | Lymph node | 7 | 0.011 | 0.13 | 30 | 0 | 0 | 0 | 0 | ABC |
| | | NOS | | | | | | | | | | | | |
| 24 | DLBCL24 | DLBCL- | 55 | F | Lymph node | 1 | 0.011 | 0.63 | 90 | | 0 | 0 | 0 | ND |
| | | NOS | | | | | | | | | | | | |
| 25 | DLBCL25 | DLBCL- | 38 | M | Tonsil | 7 | 0.008 | 0.1 | 20 | 70 | 0 | 0 | 1 | ABC |
| | | NOS | | | | | | | | | | | | |
| 26 | DLBCL26 | DLBCL- | 30 | M | Lymph node | 6 | 0.006 | 0.01 | 20 | 0 | 0 | 0 | 0 | ABC |
| | | NOS | | | | | | | | | | | | |
| 27 | DLBCL27 | DLBCL- | 78 | F | Tonsil | 7 | 0.005 | 0.01 | 20 | 100 | 0 | 0 | 0 | Type 3 |
| | | NOS | | | | | | | | | | | | |
| 28 | DLBCL28 | DLBCL- | 67 | F | Lymph node | 8 | 0.003 | 0.02 | 30 | 10 | 0 | 0 | 0 | GCB |
| | | NOS | | | | | | | | | | | | |
| 29 | DLBCL29 | DLBCL- | 57 | F | Thyroid | 5 | 0.001 | 0 | 30 | 0 | 0 | 0 | 0 | GCB |
| | | NOS | | | | | | | | | | | | |
| Failed | DLBCL30 | DLBCL- | 68 | F | Soft tissue | 8 | — | — | 80 | | 0 | 0 | 0 | ND |
| QC | | NOS | | | mass | | | | | | | | | |

^{¶}Cell of Origin (COO): * refers to COO as classified using Hans IHC criteria (Hans et al). ‘ND’ is ‘not done’.

Test Cohort: Burkitt Lymphoma

| | | | | Age of | | | Age of | | MYC | MYC | |
|---|---|---|---|---|---|---|---|---|---|---|---|
| | Figure | | | patient | | | Biopsy | Diagnostic | Activity | IHC | MYC |
| Case | Code | Diagnosis | Subtype | (years) | Sex | Site of Biopsy | (years) | Score | Score | (%) | Rearrangement* |
| 1 | tBL1 | Burkitt | Sporadic | 4 | M | Pelvic soft | 3 | 0.946 | 0.996 | 80 | 1 |
| | | Lymphoma | | | | tissue mass | | | | | |
| 2 | tBL2 | Burkitt | Sporadic | 42 | F | Thyroid Mass | 10 | 0.883 | 0.996 | 0 | 1 |
| | | Lymphoma | | | | | | | | | |
| | | (Atypical) | | | | | | | | | |
| 3 | tBL3 | Burkitt | Sporadic | 40 | F | Ovary and | 0.5 | 0.877 | 0.955 | 0 | 1 |
| | | Lymphoma | | | | fallopian tube | | | | | |
| 4 | tBL4 | Burkitt | Sporadic | 55 | M | Right | 11 | 0.845 | 0.862 | 90 | 1 |
| | | Lymphoma | | | | Mandibular | | | | | |
| | | | | | | mass | | | | | |
| 5 | tBL5 | Burkitt | Immunodeficiency | 41 | M | Lymph node | 7 | 0.811 | 0.919 | 60 | 1 |
| | | Lymphoma | | | | | | | | | |
| 6 | tBL6 | Burkitt | Sporadic | 10 | M | Nasopharyngeal | 10 | 0.802 | 0.963 | 90 | 1 |
| | | Lymphoma | | | | mass | | | | | |
| 7 | tBL7 | Burkitt | Sporadic | 47 | M | Mediastinal | 10 | 0.766 | 0.969 | 10 | 1 |
| | | Lymphoma | | | | Mass | | | | | |
| 8 | tBL8 | Burkitt | Sporadic | 33 | M | Right Neck | 7 | 0.682 | 0.991 | 80 | — |
| | | Lymphoma | | | | mass | | | | | |
| 9 | tBL9 | Burkitt | Sporadic | 44 | M | Lymph node | 9 | 0.641 | 0.975 | 90 | 1 |
| | | Lymphoma | | | | | | | | | |
| Failed | tBL10 | Burkitt | Sporadic | 27 | M | Oropharynx | 1 | — | — | 100 | 1 |
| QC | | Lymphoma | | | | | | | | | |
| Failed | tBL11 | Burkitt | Sporadic | 14 | F | Pelvic mass | 1 | — | — | 100 | 1 |
| QC | | Lymphoma | | | | | | | | | |
| Failed | tBL12 | Burkitt | Sporadic | 45 | M | Right Femur | 2 | — | — | 90 | 1 |
| QC | | Lymphoma | | | | | | | | | |

Test Cohort: Genetic Double Hit Lymphoma

| | | | Age of | | | Age of | | MYC | | | | | |
|---|---|---|---|---|---|---|---|---|---|---|---|---|---|
| | Figure | | Patient | | | Biopsy | Diagnostic | Activity | MYC IHC | BCL2 | MYC- | BCL2- | BCL- |
| Case | Code | Diagnosis | (years) | Sex | Site of Biopsy | (years) | Score | Score | (%) | IHC (%) | R* | R* | R* |
| 1 | tDHL1 | BCL-U | 55 | F | Maxilla | 3 | 0.901 | 0.981 | 100 | 0 | 1 | 1 | 0 |
| 2 | tDHL2 | BCL-U | 86 | F | Lymph Node | 3 | 0.849 | 1 | 90 | 0 | 1 | 0 | 1 |
| 3 | tDHL3 | BCL-U | 64 | M | Retroperit. | 5 | 0.773 | 0.986 | 100 | 100 | 1 | 1 | 0 |
| 4 | tDHL4 | BCL-U | 40 | F | Lymph Node | 6 | 0.305 | 0.983 | 80 | 50 | 1 | 1 | 0 |
| 5 | tDHL5 | BCL-U | 68 | F | Lymph Node | 3 | 0.119 | 0.598 | 60 | 100 | 1 | 1 | 0 |
| 6 | tDHL6 | DLBCL | 46 | M | Oropharynx | 11 | 0.046 | 0.997 | 80 | 0 | 1 | 1 | 0 |
| 7 | tDHL7 | DLBCL | 41 | M | Lymph Node | 3 | 0.02 | 0.259 | 70 | 100 | 1 | 1 | 0 |
| 8 | tDHL8 | DLBCL | 62 | F | Pancreas | 3 | 0.015 | 0.177 | 70 | 100 | 1 | 1 | 0 |

Test Cohort: DLBCL

| | | | Age of Patient | | Site of | Age of Biopsy | Diagnostic | MYC Activity | |
|---|---|---|---|---|---|---|---|---|---|
| Case | Figure Code | Diagnosis | (years) | Sex | Biopsy | (years) | Score | Score | MYC IHC (%) |
| 1 | tDLBCL1 | DLBCL, | 43 | M | Nasopharynx | 12 | 0.501 | 0.629 | 70 |
| | | NOS | | | | | | | |
| 2 | tDLBCL2 | DLBCL, | 76 | M | Mediastinum | 12 | 0.332 | 0.122 | 90 |
| | | NOS | | | | | | | |
| 3 | tDLBCL3 | DLBCL, | 58 | F | Lymph | 10 | 0.167 | 0.638 | 90 |
| | | NOS | | | Node | | | | |
| 4 | tDLBCL4 | DLBCL- | 72 | F | Clavicle | 7 | 0.15 | 0.51 | 40 |
| | | NOS | | | | | | | |
| 5 | tDLBCL5 | DLBCL- | 65 | M | Stomach | 7 | 0.09 | 0.85 | 50 |
| | | NOS | | | | | | | |
| 6 | tDLBCL6 | DLBCL, Immunoblastic | 91 | F | Mediastinum | 10 | 0.086 | 0.661 | 80 |
| 7 | tDLBCL7 | DLBCL- | 80 | M | Lymph | 8 | 0.06 | 0.38 | 40 |
| | | NOS | | | Node | | | | |
| 8 | tDLBCL8 | DLBCL, | 71 | M | Lymph | 10 | 0.062 | 0.35 | 60 |
| | | NOS | | | Node | | | | |
| 9 | tDLBCL9 | DLBCL, | 44 | F | Lung | 12 | 0.055 | 0.816 | 90 |
| | | NOS | | | | | | | |
| 10 | tDLBCL10 | DLBCL, | 41 | M | GI | 12 | 0.052 | 0.076 | 10 |
| | | HIV+ | | | Cecum | | | | |
| 11 | tDLBCL11 | DLBCL, | 78 | M | Lymph | 11 | 0.049 | 0.06 | 20 |
| | | NOS | | | Node | | | | |
| 12 | tDLBCL12 | DLBCL- | 53 | M | Lymph | 6 | 0.05 | 0.18 | 30 |
| | | NOS | | | Node | | | | |
| 13 | tDLBCL13 | DLBCL, | 54 | M | Oropharynx | 9 | 0.046 | 0.024 | 60 |
| | | NOS | | | | | | | |
| 14 | tDLBCL14 | DLBCL- | 80 | F | Spleen | 7 | 0.04 | 0.27 | 50 |
| | | NOS | | | | | | | |
| 15 | tDLBCL15 | DLBCL, | 46 | F | Kidney | 11 | 0.04 | 0.517 | 40 |
| | | NOS | | | | | | | |
| 16 | tDLBCL16 | DLBCL, | 68 | M | Lymph | 12 | 0.035 | 0.594 | 30 |
| | | NOS | | | Node | | | | |
| 17 | tDLBCL17 | DLBCL, | 73 | F | Omentum | 13 | 0.034 | 0.047 | 40 |
| | | NOS | | | | | | | |
| 18 | tDLBCL18 | DLBCL, | 44 | M | Orbit | 8 | 0.031 | 0.004 | 10 |
| | | NOS | | | | | | | |
| 19 | tDLBCL19 | DLBCL- | 77 | F | Lymph | 7 | 0.03 | 0.09 | 20 |
| | | NOS | | | Node | | | | |
| 20 | tDLBCL20 | DLBCL, | 62 | M | Spine | 11 | 0.026 | 0.032 | 20 |
| | | NOS | | | | | | | |
| 21 | tDLBCL21 | DLBCL, | 76 | M | Lymph | 13 | 0.024 | 0.121 | 20 |
| | | NOS | | | Node | | | | |
| 22 | tDLBCL22 | DLBCL- | 33 | F | Lymph | 8 | 0.02 | 0.3 | 20 |
| | | NOS | | | Node | | | | |
| 23 | tDLBCL23 | DLBCL- | 78 | F | Lymph | 7 | 0.02 | 0.46 | 40 |
| | | NOS | | | Node | | | | |
| 24 | tDLBCL24 | DLBCL, | 54 | M | Lymph | 12 | 0.016 | 0.983 | 70 |
| | | NOS | | | Node | | | | |
| 25 | tDLBCL25 | DLBCL- | 72 | M | Neck | 7 | 0.02 | 0.02 | 40 |
| | | NOS | | | Node | | | | |
| 26 | tDLBCL26 | DLBCL- | 27 | M | Lymph | 5 | 0.02 | 0.04 | 40 |
| | | NOS | | | Node | | | | |
| 27 | tDLBCL27 | DLBCL, | 58 | F | Ovary | 9 | 0.015 | 0.378 | 30 |
| | | NOS | | | | | | | |
| 28 | tDLBCL28 | DLBCL- | 69 | M | Scapula | 9 | 0.01 | 0.02 | 50 |
| | | NOS | | | lesion | | | | |
| 29 | tDLBCL29 | DLBCL- | 46 | F | Antral | 9 | 0.01 | 0.04 | 30 |
| | | NOS | | | mass | | | | |
| 30 | tDLBCL30 | DLBCL, | 49 | F | Trachea | 12 | 0.012 | 0.038 | 10 |
| | | NOS | | | | | | | |
| 31 | tDLBCL31 | DLBCL- | 32 | M | Pericardial | 6 | 0.01 | 0 | 10 |
| | | NOS | | | | | | | |
| 32 | tDLBCL32 | DLBCL, | 60 | M | Lymph | 8 | 0.008 | 0.009 | 30 |
| | | NOS | | | Node | | | | |
| 33 | tDLBCL33 | DLBCL- | 68 | M | R chest | 7 | 0.01 | 0.03 | 40 |
| | | NOS | | | wall | | | | |
| 34 | tDLBCL34 | DLBCL- | 69 | F | Mesenteric | 9 | 0.01 | 0 | — |
| | | NOS | | | | | | | |
| 35 | tDLBCL35 | DLBCL- | 40 | M | Extradural | 8 | 0 | 0.003 | 40 |
| | | NOS | | | | | | | |
| 36 | tDLBCL36 | DLBCL- | 59 | F | Lymph | 9 | 0 | 0.27 | 50 |
| | | NOS | | | Node | | | | |
| 37 | tDLBCL37 | DLBCL- | 62 | M | Spleen | 4 | 0 | 0.1 | 40 |
| | | NOS | | | | | | | |
| 38 | tDLBCL38 | DLBCL- | 79 | F | Lymph | 6 | 0 | 0 | — |
| | | NOS | | | Node | | | | |
| (39) | tDHL6 | DLBCL^{¶} | 46 | M | Oropharynx | 11 | 0.046 | 0.997 | 80 |
| (40) | tDHL7 | DLBCL^{¶} | 41 | M | Lymph | 3 | 0.02 | 0.259 | 70 |
| | | | | | Node | | | | |
| (41) | tDHL8 | DLBCL^{¶} | 62 | F | Pancreas | 3 | 0.015 | 0.177 | 70 |
| Failed | tDLBCL39 | DLBCL, | 72 | M | Lymph | 11 | — | — | — |
| QC | | NOS | | | Node | | | | |

| Case | BCL2 IHC (%) | MYC-R* | BCL2-R* | BCL6-R* | CD5 IHC | EBV (EBERs) | COO⁺ |
|---|---|---|---|---|---|---|---|
| 1 | 0 | 1 | 0 | 0 | 0 | 0 | GCB* |
| 2 | 80 | 0 | 0 | — | 0 | 0 | Non- |
| | | | | | | | GCB* |
| 3 | 100 | 0 | — | — | 0 | 0 | GCB* |
| 4 | 0 | 0 | 0 | 0 | 0 | 0 | Type 3 |
| 5 | 0 | 0 | 0 | 0 | 0 | 0 | ND |
| 6 | 100 | 0 | 0 | 0 | 0 | 0 | GCB* |
| 7 | 10 | 0 | 0 | 0 | 0 | 0 | ABC |
| 8 | 100 | — | 0 | 0 | 0 | 0 | GCB* |
| 9 | 100 | 0 | 0 | — | 0 | 0 | Non- |
| | | | | | | | GCB* |
| 10 | 0 | 0 | 0 | 0 | 1 | 0 | GCB* |
| 11 | 60 | 0 | 0 | 0 | 0 | 0 | GCB* |
| 12 | 20 | 0 | — | — | 1 | 1 | ABC |
| 13 | 10 | 0 | 0 | 0 | 0 | 0 | GCB* |
| 14 | 0 | 0 | 0 | 0 | 0 | 0 | ABC |
| 15 | 10 | 0 | 0 | 0 | 0 | 0 | Non- |
| | | | | | | | GCB* |
| 16 | 90 | 0 | — | — | 1 | 0 | Non- |
| | | | | | | | GCB* |
| 17 | 80 | 0 | 1 | 0 | 0 | 0 | Non- |
| | | | | | | | GCB* |
| 18 | 0 | 0 | 0 | 0 | 0 | 0 | GCB* |
| 19 | 100 | 0 | — | — | 0 | 0 | Type 3 |
| 20 | 100 | 0 | 0 | 0 | 0 | 0 | Non- |
| | | | | | | | GCB* |
| 21 | 0 | 0 | 0 | 1 | 0 | 0 | GCB* |
| 22 | 0 | 0 | 0 | 1 | 0 | 0 | ABC |
| 23 | 30 | 0 | 0 | 0 | 0 | 0 | ABC |
| 24 | 100 | 0 | 0 | | 0 | 0 | GCB* |
| 25 | 90 | 0 | — | — | 0 | 0 | GCB |
| 26 | 80 | 0 | 0 | 0 | 0 | 0 | ABC |
| 27 | 90 | 0 | 0 | 1 | 0 | 0 | Non- |
| | | | | | | | GCB* |
| 28 | 0 | 0 | — | — | 0 | 0 | GCB |
| 29 | 50 | 0 | — | — | 0 | 0 | ND |
| 30 | 90 | 0 | 0 | 0 | 1 | 0 | Non- |
| | | | | | | | GCB* |
| 31 | 100 | 0 | 0 | 0 | 0 | 0 | GCB |
| 32 | 30 | 0 | 0 | 1 | 0 | 0 | GCB* |
| 33 | 30 | 0 | — | — | 0 | 0 | ABC |
| 34 | 90 | 0 | 0 | 0 | 0 | 0 | Type 3 |
| 35 | 0 | 0 | 0 | 0 | 0 | 0 | GCB |
| 36 | 90 | 0 | 0 | 0 | 0 | 0 | ABC |
| 37 | 0 | 0 | 0 | 0 | 0 | 0 | GCB |
| 38 | 0 | 0 | 0 | 0 | 0 | 0 | ND |
| (39) | 0 | 1 | 1 | 0 | 0 | 0 | GCB* |
| (40) | 100 | 1 | 1 | 0 | 0 | 0 | ND |
| (41) | 100 | 1 | 1 | 0 | 0 | 0 | ND |
| Failed | 80 | 0 | 0 | 0 | 0 | 0 | ND |
| QC | | | | | | | |

*For all references to MYC-, BCL2- and BCL6-rearrangement, “1” denotes confirmed rearrangement, “0” rearrangement not tested, “NA” not available
^{¶}Genetic double-hit lymphoma with a pathological diagnosis of DLBCL are included here as well as in the ‘Genetic Double Hit Lymphoma’ table
⁺Cell of Origin (COO): * refers to COO as classified using Hans IHC criteria (Hans et al). ‘ND’ is ‘not done’.

Immunohistochemistry and Cytogenetic Analyses

MYC IHC was performed on 96 tumors using a rabbit monoclonal antibody (clone Y69, Epitomics/Abcam, cat. #ab32072) as described¹⁵. The status of the MYC locus was determined by fluorescence in situ hybridization (FISH) analysis for 96 tumors using Vysis LSI MYC “break-apart” probe set (cat. #05491-001), as described¹⁵. FISH analyses were performed on indicated cases using the BCL2-IgH dual fusion (cat. #05-J71-001), and BCL6-IgH “break-apart” (cat. #01N23-020; Abbott Laboratories, Abbott Park, Ill.) probe sets respectively, following manufacturer's recommendations. For a minority of cases, a karyotype was obtained as part of the original diagnostic evaluation¹⁵.

RNA Extraction and Profiling

FFPE tissue blocks were sectioned immediately prior to the RNA extraction. For each block, the initial 10 μm section was discarded and 3×10 μm subsequent sections were taken for analysis. If the estimated surface area of lesional tissue was <5 mm² an extra 10 μm section was taken. Total RNA was isolated using the Qiagen RNeasy kit (catalog #73504, Qiagen, Hilden, Germany) and quantified using Nanodrop spectrophotometry (Nanodrop Products, Thermo Science, DE). RNA was diluted to 150-200 ng/5 μL, aliquoted and stored at −80° C. until use.

For the multiplexed, digital gene expression analysis, 150-200 ng of RNA for each sample was hybridized with 20 μl of reporter probes/reaction buffer and 5 μl of capture probes at 65° C. for 20 hours. The hybridized samples were then processed on the NanoString nCounter preparation station for 2.5 hours and expression data were subsequently generated on the NanoString nCounter digital analyzer (NanoString Technologies, Seattle, Wash.) using the 600 fields of view setting over 4 hours³⁰. In total tumors from 96 patients were profiled, with a further 5 tumors (5%) failing analytical quality control.

Target Selection for the Initial and Final Profiling Panels

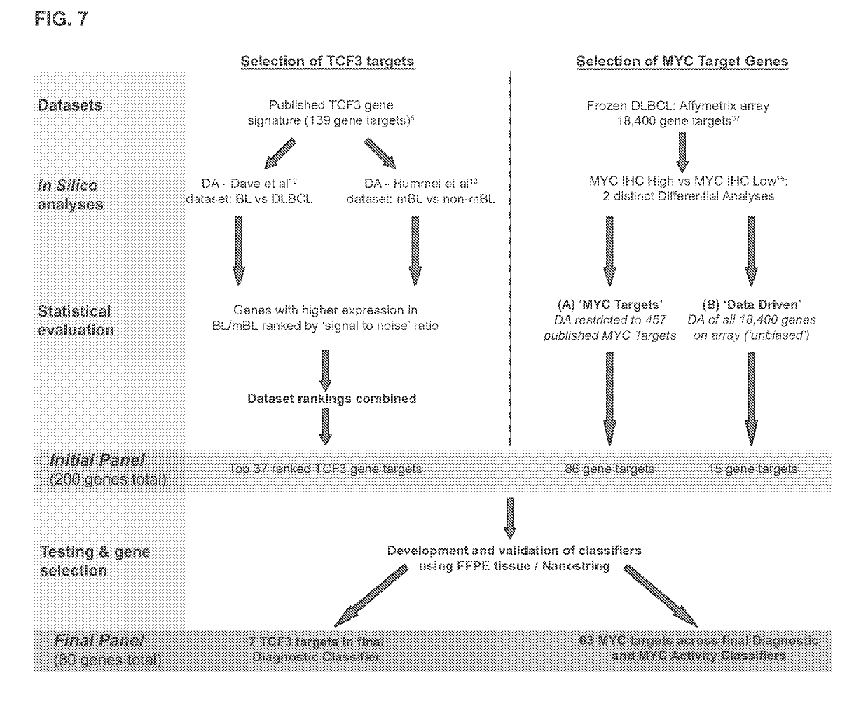

Candidate gene targets were initially selected from published GEPs of BL and DLBCL^{12,13} with preference given to genes within the TCF3/ID3 signaling pathway⁵, published MYC targets³¹⁻³⁷, and GEPs of frozen tissue corresponding to DLBCL samples in the training set³⁸. These were supplemented by additional targets of interest including housekeeping genes (FIG. 7).

The initial panel of 200 probes included 37 unique transcripts distilled from a previously published “TCF3 signature”⁵. These were subsequently validated, by in silico differential analysis (DA), as best distinguishing BL from DLBCL in two independent series of B-cell non-Hodgkin lymphomas^{12,13} (FIG. 7). The panel also included transcripts from 7 published datasets of MYC targets (101 targets selected)³¹⁻³⁷ that were validated (False Discovery rate (FDR)<0.25; fold change (FC)>1.3) by DA against Affymetrix U133 microarray GEPs of frozen DLBCLs with corresponding MYC IHC scores from matched FFPE tissue in the training cohort^{15,38} and differentially expressed genes suggested by DA of the GEPs of frozen DLBCLs with corresponding MYC IHC scores (FDR<0.25; FC>2.0). Finally they were supplemented with BCL2 and related family members (5 targets), “housekeeping” control transcripts (15 targets), and select markers of specific cell lineages (CD3e, CD68, CD19, CD79a, CD20; Table 2).

The final profiling panel, targeting 80 transcripts, was derived by analyzing data from the training set, both ranking the importance of each included gene and estimating how could exclude many without compromising the predicted accuracy (Table 3). The predicted accuracy of each classifier was assessed on the training set using leave-one-out cross-validation (LOO-CV), as well as on an independent test dataset.

TABLE 2
200-gene Initial Profiling panel. The 200 genes targets used in the initial
profiling panel are listed and are organized into groups as derived
(see FIG. 7 and methods). ‘Data driven’ targets are genes that were not
previously published as MYC targets but that were differentially expressed
in the training set in either MYC IHC-High or MYC IHC-Low DLBCL.

| | | |
|---|---|---|
| | | Target |
| | Accession | Region |
| Gene Symbol | Number | (base pairs) |

Table 2 - Published MYC Targets

| | | | |
|---|---|---|---|
| 1 | ACHY | NM_000687.2 | 1805-1905 |
| 2 | AKAP1 | NM_139275.1 | 2725-2825 |
| 3 | AMD1 | NM_001634.4 | 810-910 |
| 4 | APEX1 | NM_001641.2 | 727-827 |
| 5 | APITD1 | NM_199294.2 | 950-1050 |
| 6 | AURKA | NM_003600.2 | 405-505 |
| 7 | BUB1B | NM_001211.4 | 835-935 |
| 8 | FAM216A/C12ORF24 | NM_013300.2 | 722-822 |
| 9 | CCNB1 | NM_031966.2 | 715-815 |
| 10 | CDC25A | NM_001789.2 | 690-790 |
| 11 | CDK4 | NM_000075.2 | 1055-1155 |
| 12 | CHN1 | NM_001025201.2 | 1965-2065 |
| 13 | CIRH1A | NM_032830.2 | 84-184 |
| 14 | CTPS | NM_001905.2 | 2570-2670 |
| 15 | CYCS | NM_018947.4 | 1735-1835 |
| 16 | DDX21 | NM_004728.2 | 685-785 |
| 17 | DDX47 | NM_016355.3 | 1180-1280 |
| 18 | DHX33 | NM_001199699.1 | 2873-2973 |
| 19 | DKC1 | NM_001363.3 | 2255-2355 |
| 20 | DLEU1 | NR_002605.1 | 173-273 |
| 21 | EIF1AX | NM_001412.3 | 3818-3918 |
| 22 | ETFA | NM_001127716.1 | 630-730 |
| 23 | EXOSC8 | NM_181503.1 | 655-755 |
| 24 | FBL | NM_001436.3 | 883-983 |
| 25 | FKBP4 | NM_002014.3 | 2755-2855 |
| 26 | FXN | NM_001161706.1 | 515-615 |
| 27 | GEMIN4 | NM_015721.2 | 1925-2025 |
| 28 | GEMIN5 | NM_015465.3 | 4760-4860 |
| 29 | GINS2 | NM_016095.2 | 990-1090 |
| 30 | GOT2 | NM_002080.2 | 2145-2245 |
| 31 | GPD1L | NM_015141.2 | 2565-2665 |
| 32 | HSPE1 | NM_002157.2 | 65-165 |
| 33 | IDH3A | NM_005530.2 | 1521-1621 |
| 34 | IMPA2 | NM_014214.1 | 545-645 |
| 35 | KIAA0101 | NM_014736.4 | 65-165 |
| 36 | LDHB | NM_001174097.1 | 1190-1290 |
| 37 | LMNB2 | NM_032737.2 | 3630-3730 |
| 38 | LRPPRC | NM_133259.3 | 6220-6320 |
| 39 | LSM7 | NM_016199.2 | 150-250 |
| 40 | LYAR | NM_001145725.1 | 230-330 |
| 41 | MCC | NM_001085377.1 | 5578-5678 |
| 42 | MDM1 | NM_017440.2 | 1360-1460 |
| 43 | MGST1 | NM_145764.1 | 330-430 |
| 44 | MKI67IP | NM_032390.4 | 215-315 |
| 45 | MRPS2 | NR_051968.1 | 1512-1612 |
| 46 | MRPS34 | NM_023936.1 | 719-819 |
| 47 | MYB | NM_005375.2 | 3145-3245 |
| 48 | MYC | NM_002467.3 | 1610-1710 |
| 49 | NAP1L1 | NM_004537.4 | 543-643 |
| 50 | NME1 | NM_000269.2 | 500-600 |
| 51 | NOLC1 | NM_004741.3 | 3405-3505 |
| 52 | NOP2 | NM_001033714.1 | 1800-1900 |
| 53 | NPM1 | NM_002520.5 | 10-110 |
| 54 | NUDCD2 | NM_145266.4 | 368-468 |
| 55 | PA2G4 | NM_006191.2 | 2475-2575 |
| 56 | PAICS | NM_001079524.1 | 2604-2704 |
| 57 | PDHA1 | NM_000284.3 | 1080-1180 |
| 58 | PDLIM3 | NM_014476.4 | 897-997 |
| 59 | PHB | NM_002634.2 | 1270-1370 |
| 60 | PHB2 | NM_007273.3 | 1210-1310 |
| 61 | POLR3K | NM_016310.2 | 395-495 |
| 62 | PPAT | NM_002703.3 | 1210-1310 |
| 63 | PPRC1 | NM_015062.3 | 4640-4740 |
| 64 | PRMT1 | NM_001536.4 | 444-544 |
| 65 | PSMG1 | NM_203433.1 | 655-755 |
| 66 | RAB8B | NM_016530.2 | 4157-4257 |
| 67 | RANBP1 | NM_002882.2 | 380-480 |
| 68 | RFC3 | NM_002915.3 | 740-840 |
| 69 | RIN2 | NM_018993.2 | 690-790 |
| 70 | RPIA | NM_144563.2 | 1588-1688 |
| 71 | RPL22 | NM_000983.3 | 1270-1370 |
| 72 | RPL23 | NM_000978.3 | 71-171 |
| 73 | RRS1 | NM_015169.3 | 1247-1347 |
| 74 | SFRS7 | NM_001031684.2 | 532-632 |
| 75 | SRM | NM_003132.2 | 254-354 |
| 76 | SSBP1 | NM_003143.1 | 235-335 |
| 77 | STRAP | NM_007178.3 | 1535-1635 |
| 78 | STRBP | NM_001171137.1 | 1150-1250 |
| 79 | TFDP1 | NM_007111.4 | 1826-1926 |
| 80 | TIPIN | NM_017858.2 | 230-330 |
| 81 | TMEM97 | NM_014573.2 | 2055-2155 |
| 82 | TRAP1 | NM_016292.2 | 1293-1393 |
| 83 | TYMS | NM_001071.1 | 555-655 |
| 84 | UBE2CBP (UBE3D) | NM_198920.1 | 834-934 |
| 85 | UCHL3 | NM_006002.3 | 375-475 |
| 86 | WDR3 | NM_006784.2 | 90-190 |

Table 2 - Additional Published

| | | | |
|---|---|---|---|
| 1 | ABCE1 | NM_001040876.1 | 635-735 |
| 2 | AIMP2 | NM_006303.3 | 507-607 |
| 3 | BRD2 | NM_005104.2 | 1890-1990 |
| 4 | BRD3 | NM_007371.3 | 2645-2745 |
| 5 | BRD4 | NM_014299.2 | 745-845 |
| 6 | CAD | NM_004341.3 | 2380-2480 |
| 7 | CD44 | NM_001001392.1 | 429-529 |
| 8 | CDK4 | NM_000075.2 | 1055-1155 |
| 9 | EBNA1BP2 | NM_006824.2 | 318-418 |
| 10 | EEF1A2 | NM_001958.2 | 1045-1145 |
| 11 | EXOSC8 | NM_181503.1 | 655-755 |
| 12 | FASN | NM_004104.4 | 5387-5487 |
| 13 | HNRNPA2B1 | NM_002137.3 | 435-535 |
| 14 | IARS | NM_002161.3 | 3952-4052 |
| 15 | LDHA | NM_001165414.1 | 1690-1790 |
| 16 | LRP8 | NM_033300.2 | 1590-1690 |
| 17 | MAT2A | NM_005911.4 | 805-905 |
| 18 | MAX | NM_002382.3 | 240-340 |
| 19 | MITF | NM_000248.3 | 3240-3340 |
| 20 | MRPL3 | NM_007208.2 | 350-450 |
| 21 | MYCL1 | NM_001033081.2 | 568-668 |
| 22 | MYCN | NM_005378.4 | 1545-1645 |
| 23 | NCL | NM_005381.2 | 1492-1592 |
| 24 | p50 (NFKB1) | NM_003998.2 | 1675-1775 |
| 25 | p65 (GORASP1) | NM_031899.2 | 2755-2855 |
| 26 | PEBP1 | NM_002567.2 | 1335-1435 |
| 27 | POLD2 | NM_006230.1 | 505-605 |
| 28 | POLR2H | NM_006232.2 | 317-417 |
| 29 | PRDX4 | NM_006406.1 | 540-640 |
| 30 | PYCR1 | NM_006907.2 | 513-613 |
| 31 | RPL23 | NM_000978.3 | 71-171 |
| 32 | RRP1B | NM_015056.2 | 1070-1170 |
| 33 | SLC16A1 | NM_003051.3 | 635-735 |
| 34 | SLC39A14 | NM_001128431.2 | 1245-1345 |
| 35 | SLC39A6 | NM_012319.2 | 1580-1680 |
| 36 | TBL3 | NM_006453.2 | 1070-1170 |
| 37 | UCK2 | NM_012474.3 | 730-830 |

Table 2 - Data-driven MYC High

| | | | |
|---|---|---|---|
| 1 | KIAA1737 | NM_033426.2 | 3868-3968 |
| 2 | FAM211A-AS1/C17orf76-AS1 | NR_027164.1 | 214-314 |
| 3 | PCDH9 | NM_020403.3 | 3580-3680 |
| 4 | SAMD13 | NM_001010971.2 | 672-772 |
| 5 | SERHL2 | NM_014509.3 | 637-737 |
| 6 | TCL1A | NM_001098725.1 | 867-967 |
| 7 | TMEM100 | NM_018286.2 | 655-755 |

Table 2 - Data-driven MYC Low

| | | | |
|---|---|---|---|
| 1 | SHISA8 | NM_001207020.1 | 1111-1211 |
| 2 | EGFL6 | NM_015507.2 | 1495-1595 |
| 3 | IGFBP2 | NM_000597.2 | 675-775 |
| 4 | P2RY12 | NM_022788.3 | 230-330 |
| 5 | SLAMF1 | NM_003037.2 | 580-680 |
| 6 | SLC12A8 | NM_024628.5 | 770-870 |
| 7 | TDO2 | NM_005651.1 | 0-100 |
| 8 | TMEM119 | NM_181724.2 | 1490-1590 |

Table 2 - TCF3

| | | | |
|---|---|---|---|
| 1 | ALDH5A1 | NM_001080.3 | 455-555 |
| 2 | ATF4 | NM_001675.2 | 1151-1251 |
| 3 | BMP7 | NM_001719.1 | 525-625 |
| 4 | KIAA0226L/C13orf18 | NM_025113.2 | 1071-1171 |
| 5 | CBFA2T3 | NM_005187.5 | 3195-3295 |
| 6 | CCRL1 | NM_178445.1 | 2200-2300 |
| 7 | CD38 | NM_001775.2 | 1035-1135 |
| 8 | CXCR4 | NM_003467.2 | 1335-1435 |
| 9 | NSG1/D4S234E | NM_014392.3 | 1860-1960 |
| 10 | DNMT3B | NM_175850.1 | 1950-2050 |
| 11 | DVL2 | NM_004422.2 | 1025-1125 |
| 12 | DYRK3 | NM_003582.2 | 1310-1410 |
| 13 | E2F2 | NM_004091.2 | 3605-3705 |
| 14 | FUT1 | NM_000148.3 | 3660-3760 |
| 15 | GPLD1 | NM_001503.2 | 465-565 |
| 16 | GRAP | NM_006613.3 | 1918-2018 |
| 17 | ICOSLG | NM_015259.4 | 1190-1290 |
| 18 | ID3 | NM_002167.3 | 195-295 |
| 19 | IGLL1 | NM_020070.2 | 188-288 |
| 20 | LHFP | NM_005780.2 | 460-560 |
| 21 | LZTS1 | NM_021020.2 | 3970-4070 |
| 22 | MME | NM_000902.2 | 5059-5159 |
| 23 | MRPS35 | NM_021821.2 | 250-350 |
| 24 | N4BP3 | NM_015111.1 | 5435-5535 |
| 25 | NEIL1 | NM_024608.2 | 1675-1775 |
| 26 | NEIL3 | NM_018248.2 | 842-942 |
| 27 | PPM1A | NM_021003.4 | 550-650 |
| 28 | PPP2R5C | NM_002719.3 | 1240-1340 |
| 29 | PRKAR2B | NM_002736.2 | 1350-1450 |
| 30 | RAPGEF5 | NM_012294.3 | 3420-3520 |
| 31 | RIMS3 | NM_014747.2 | 3580-3680 |
| 32 | SLC1A4 | NM_003038.4 | 3030-3130 |
| 33 | SYNE2 | NM_182914.2 | 20435-20535 |
| 34 | TBC1D1 | NM_001253915.1 | 1926-2026 |
| 35 | TCF3 | NM_003200.2 | 4325-4425 |
| 36 | TNFSF8 | NM_001244.3 | 518-618 |
| 37 | YPEL1 | NM_013313.3 | 2270-2370 |

Table 2 - BCL2

| | | | |
|---|---|---|---|
| 1 | BCL-W/BCL2L2 | NM_004050.2 | 2300-2400 |
| 2 | BCL2 | NM_000657.2 | 5-105 |
| 3 | BCL2A1 | NM_004049.2 | 80-180 |
| 4 | BCL2L1 | NM_138578.1 | 1560-1660 |
| 5 | MCL1 | NM_021960.3 | 1260-1360 |

Table 2 - Lineage-specific (‘Constitutional’)

| | | | |
|---|---|---|---|
| 1 | CD3E | NM_000733.2 | 75-175 |
| 2 | CD19 | NM_001770.4 | 1770-1870 |
| 3 | CD20/MS4A1 | NM_152866.2 | 620-720 |
| 4 | CD68 | NM_001251.2 | 1140-1240 |
| 5 | CD79A | NM_001783.3 | 695-795 |

Table 2 - Housekeeping Genes

| | | | |
|---|---|---|---|
| 1 | AAMP | NM_001087.3 | 1646-1746 |
| 2 | ACTB | NM_001101.2 | 1010-1110 |
| 3 | FTL | NM_000146.3 | 85-185 |
| 4 | GAPDH | NM_002046.3 | 972-1072 |
| 5 | GNB2L1 | NM_006098.4 | 375-475 |
| 6 | H3F3A | NM_002107.3 | 190-290 |
| 7 | HMBS | NM_000190.3 | 315-415 |
| 8 | KARS | NM_005548.2 | 1885-1985 |
| 9 | PPIA (Cyclophyllin A) | NM_021130.2 | 925-1025 |
| 10 | PSMB3 | NM_002795.2 | 340-440 |
| 11 | PSMD2 | NM_002808.3 | 771-871 |
| 12 | PTDSS1 | NM_014754.1 | 2375-2475 |
| 13 | TBP | NM_001172085.1 | 587-687 |
| 14 | TCFL1 (VPS72) | NM_005997.1 | 1112-1212 |
| 15 | TUBB | NM_178014.2 | 320-420 |

TABLE 3
80-gene Final Profiling Panel. The 80 gene targets used in the final
profiling panel are listed and are organized into groups based on
biological pathways.

| | | |
|---|---|---|
| | | Target Region |
| Gene Symbol | Accession Number | (base pairs) |

Table 3 - Published MYC Targets

| | | | |
|---|---|---|---|
| 1 | AHCY | NM_000687.2 | 1805-1905 |
| 2 | AKAP1 | NM_139275.1 | 2725-2825 |
| 3 | APEX1 | NM_001641.2 | 727-827 |
| 4 | APITD1 | NM_199294.2 | 950-1050 |
| 5 | BUB1B | NM_001211.4 | 835-935 |
| 6 | FAM216A/C12ORF24 | NM_013300.2 | 722-822 |
| 7 | CDC25A | NM_001789.2 | 690-790 |
| 8 | CDK4 | NM_000075.2 | 1055-1155 |
| 9 | CIRH1A | NM_032830.2 | 84-184 |
| 10 | CTPS | NM_001905.2 | 2570-2670 |
| 11 | DDX21 | NM_004728.2 | 685-785 |
| 12 | DHX33 | NM_001199699.1 | 2873-2973 |
| 13 | DLEU1 | NR_002605.1 | 173-273 |
| 14 | ETFA | NM_001127716.1 | 630-730 |
| 15 | FBL | NM_001436.3 | 883-983 |
| 16 | GEMIN4 | NM_015721.2 | 1925-2025 |
| 17 | GOT2 | NM_002080.2 | 2145-2245 |
| 18 | KIAA0101 | NM_014736.4 | 65-165 |
| 19 | LDHB | NM_001174097.1 | 1190-1290 |
| 20 | LYAR | NM_001145725.1 | 230-330 |
| 21 | MRPS2 | NR_051968.1 | 1512-1612 |
| 22 | MRPS34 | NM_023936.1 | 719-819 |
| 23 | MYC | NM_002467.3 | 1610-1710 |
| 24 | NME1 | NM_000269.2 | 500-600 |
| 25 | NOLC1 | NM_004741.3 | 3405-3505 |
| 26 | NOP2 | NM_001033714.1 | 1800-1900 |
| 27 | PAICS | NM_001079524.1 | 2604-2704 |
| 28 | PHB | NM_002634.2 | 1270-1370 |
| 29 | PHB2 | NM_007273.3 | 1210-1310 |
| 30 | PPAT | NM_002703.3 | 1210-1310 |
| 31 | PPRC1 | NM_015062.3 | 4640-4740 |
| 32 | PRMT1 | NM_001536.4 | 444-544 |
| 33 | RANBP1 | NM_002882.2 | 380-480 |
| 34 | RFC3 | NM_002915.3 | 740-840 |
| 35 | RRS1 | NM_015169.3 | 1247-1347 |
| 36 | SRM | NM_003132.2 | 254-354 |
| 37 | SSBP1 | NM_003143.1 | 235-335 |
| 38 | STRBP | NM_001171137.1 | 1150-1250 |
| 39 | TMEM97 | NM_014573.2 | 2055-2155 |
| 40 | TRAP1 | NM_016292.2 | 1293-1393 |
| 41 | UCHL3 | NM_006002.3 | 375-475 |
| 42 | WDR3 | NM_006784.2 | 90-190 |

Table 3 - Selected as MYC Targets

| | | | |
|---|---|---|---|
| 1 | CAD | NM_004341.3 | 2380-2480 |
| 2 | EBNA1BP2 | NM_006824.2 | 318-418 |
| 3 | FASN | NM_004104.4 | 5387-5487 |
| 4 | LRP8 | NM_033300.2 | 1590-1690 |
| 5 | NCL | NM_005381.2 | 1492-1592 |
| 6 | POLD2 | NM_006230.1 | 505-605 |
| 7 | PYCR1 | NM_006907.2 | 513-613 |
| 8 | SLC16A1 | NM_003051.3 | 635-735 |
| 9 | UCK2 | NM_012474.3 | 730-830 |

Table 3 - Data Driven MYC High

| | | | |
|---|---|---|---|
| 1 | FAM211A-AS1/ | NR_027164.1 | 214-314 |
| | C17orf76-AS1 | | |
| 2 | KIAA0226L | NM_025113.2 | 1071-1171 |
| 3 | PCDH9 | NM_020403.3 | 3580-3680 |
| 4 | SAMD13 | NM_001010971.2 | 672-772 |
| 5 | TCL1A | NM_001098725.1 | 867-967 |

Table 3 - Data Driven MYC Low

| | | | |
|---|---|---|---|
| 1 | SHISA8 | NM_001207020.1 | 1111-1211 |
| 2 | IGFBP2 | NM_000597.2 | 675-775 |
| 3 | P2RY12 | NM_022788.3 | 230-330 |
| 4 | SLAMF1 | NM_003037.2 | 580-680 |
| 5 | SLC12A8 | NM_024628.5 | 770-870 |
| 6 | TDO2 | NM_005651.1 | 0-100 |
| 7 | TMEM119 | NM_181724.2 | 1490-1590 |

Table 3 - TCF3 Targets

| | | | |
|---|---|---|---|
| 1 | BMP7 | NM_001719.1 | 525-625 |
| 2 | DNMT3B | NM_175850.1 | 1950-2050 |
| 3 | E2F2 | NM_004091.2 | 3605-3705 |
| 4 | LZTS1 | NM_021020.2 | 3970-4070 |
| 5 | MME | NM_000902.2 | 5059-5159 |
| 6 | PRKAR2B | NM_002736.2 | 1350-1450 |
| 7 | TCF3 | NM_003200.2 | 4325-4425 |

Table 3 - BCL2 Family

| | | | |
|---|---|---|---|
| 1 | BCL2 | NM_000657.2 | 5-105 |
| 2 | BCL2A1 | NM_004049.2 | 80-180 |

Table 3 - Additional Published

| | | | |
|---|---|---|---|
| 1 | CD44 | NM_001001392.1 | 429-529 |
| 2 | p50 (NFKB1) | NM_003998.2 | 1675-1775 |

Table 3 - Housekeeping Genes

| | | | |
|---|---|---|---|
| 1 | AAMP | NM_001087.3 | 1646-1746 |
| 2 | H3F3A | NM_002107.3 | 190-290 |
| 3 | HMBS | NM_000190.3 | 315-415 |
| 4 | KARS | NM_005548.2 | 1885-1985 |
| 5 | PSMB3 | NM_002795.2 | 340-440 |
| 6 | TUBB | NM_178014.2 | 320-420 |

Housekeeping Gene Transcripts

Six housekeeping (HK) genes were selected based on the following criteria: i) low variation across samples; ii) even coverage along the expression range; iii) exclusion of the most highly expressed HK genes, since at very high levels, the variation level of the HK genes is comparable to the variation of the other genes, and iv) exclusion of genes within regions of known recurrent copy number alteration in lymphoma³⁸. Based on these criteria, we selected the following 6 gene targets: AAMP, HMBS, KARS, PSMB3, TUBB, and H3F3A.

Data Normalization

Data from the preliminary targeted profiling panel (200 genes) and the final profiling panel (80 genes) were cross-normalized using expression data from 6 cases tested with both panels. Normalization of the NanoString data was performed using the R package NanoStringNorm (Waggett et al., Bioinformatics. 2012 Jun. 1; 28(11):1546-8). We used the sum of the expression values to estimate the technical assay variation, the mean to estimate background count levels and the sum of the six housekeeping genes to normalize for the RNA sample content. Additionally, the data were log 2 transformed.

Unsupervised Clustering of the Normalized Training Dataset

Unsupervised clustering of the data derived from the training set was performed using Gene-e (Broad institute; Gould J (2013). GENE.E: Interact with GENE-E from R. R package version 1.6.0). These data were normalized using Nanostring's ‘nSolver’ software package and then transformed to Log 2 using Gene-e prior to ‘one minus Pearson’ hierarchical clustering.

Classification Models

Classification models were selected based on the training cohort using a bootstrapping scheme, where 75% of the samples were drawn to train a classification model, which was then tested on the remaining 25% of the samples, with the train/test split repeated 100 times. Elastic nets³⁹, linear and polynomial support vector machines (SMV), shrunken centroids⁴⁰ and a random forest algorithm⁴¹ were evaluated as candidate prediction models. An elastic net³⁹ prediction model was selected for both classifiers, based on a bootstrapping evaluation scheme on the training set. For the development of the diagnostic classifier, cases with a pathological diagnosis of BL and DLBCL were used. For the MYC activity classifier, only DLBCLs were used, excluding BLs. DLBCLs with MYC IHC>50% and ≦50% were classified as MYC IHC-High and IHC-Low, respectively, and these labels were used in the training of the MYC activity classifier¹⁵.

Features were selected based on differential expression, and their number determined based on LOO-CV performed on the training cohort; based on this procedure, 21 genes were used for the diagnostic classifier and 61 for the MYC classifier (Table 4). Based on their performance on the training dataset we selected the elastic net with an alpha parameter of 0.1 and a lambda of 0.1 as the classifier of choice for both stages. Classification accuracy of the final elastic net models was assessed on the training cohort using LOO-CV and comparing the predictions with the outcome of the IHC staining. Unbiased validation was then performed by training elastic net models on the entire training dataset and applying them to the classification of cases in the test cohort.

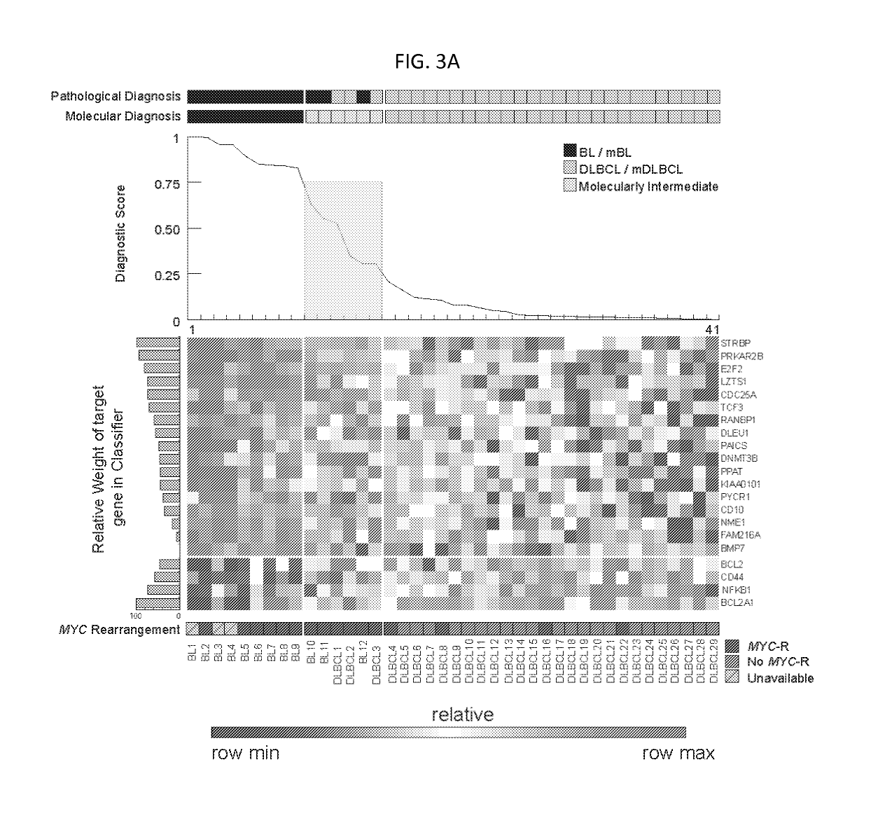

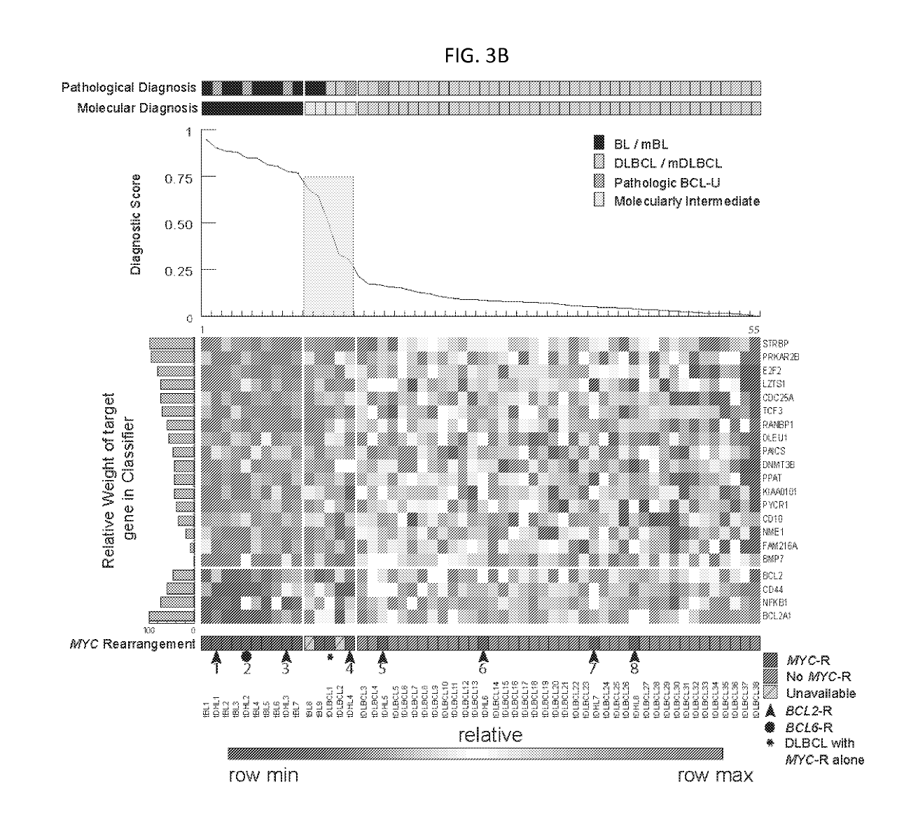

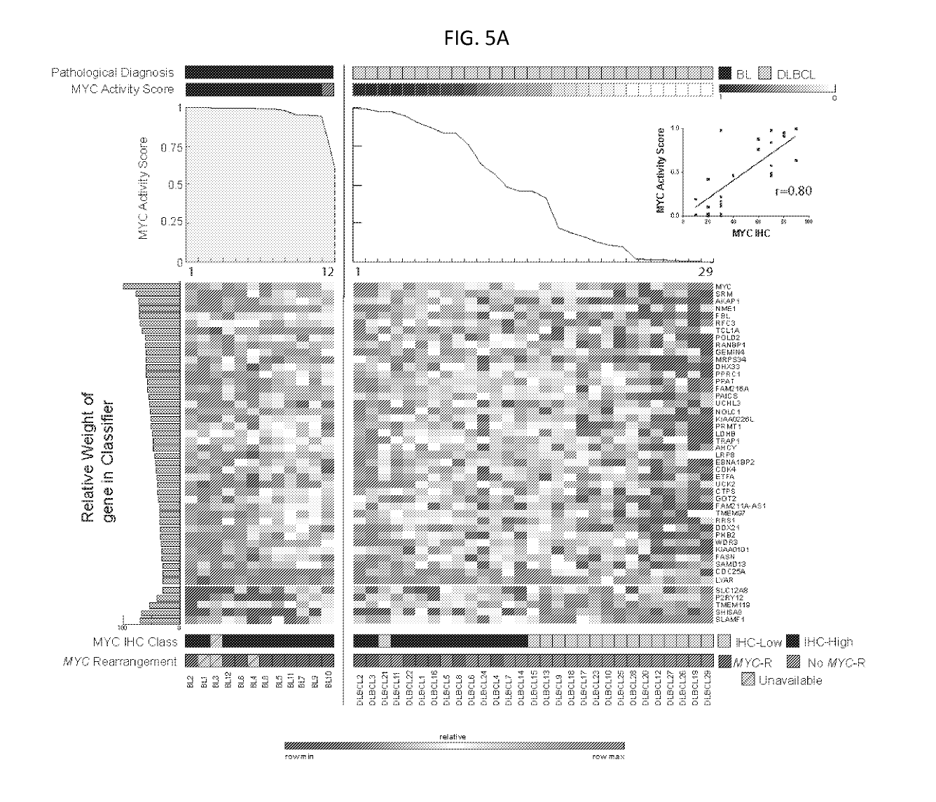

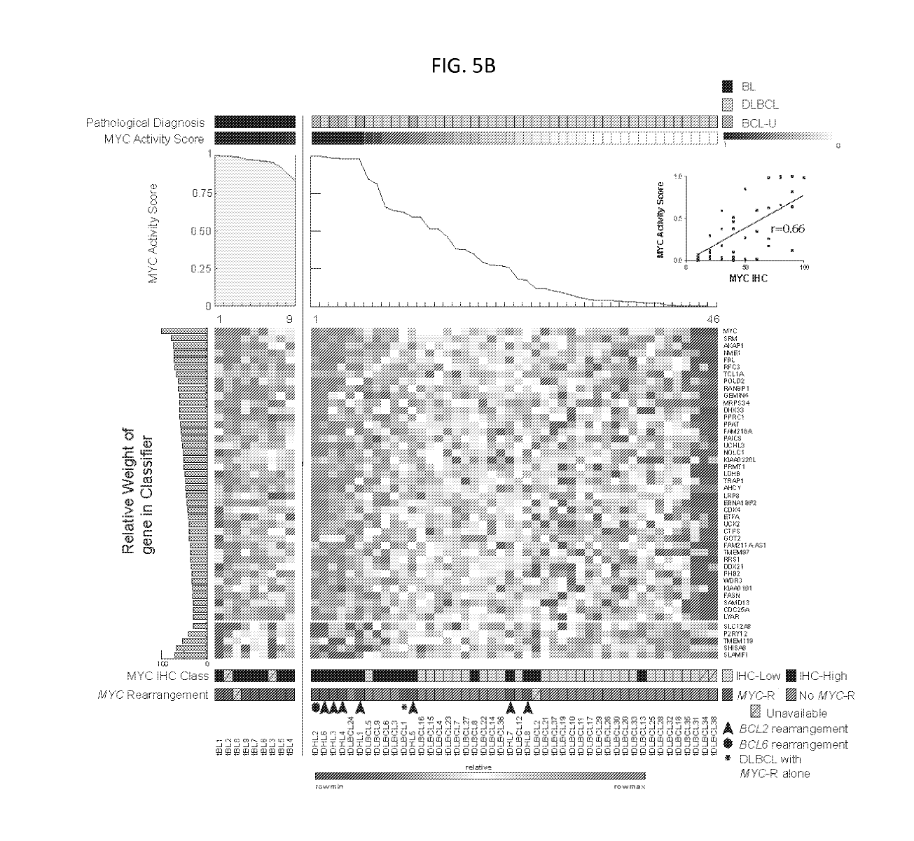

TABLE 4
The final signature genes comprising the Diagnostic Classifier (21 signature
genes) and the MYC Activity Classifier (61 signature genes) are listed, together with the
‘relative weight’ (variable importance) of each gene in the classifier, as shown in FIGS. 3A,
B and FIGS. 5A, B (see methods). Eight genes (indicated in bold) are used in both
classifiers. Housekeeping genes (6) used to normalize the datasets.

| | | Target | Variable | |
|---|---|---|---|---|
| | Accession | Region | Importance | |
| Gene Symbol | Number | (bps) | (0-100) | Gene weights |

Table 4 - Diagnostic Classifier

| | | | | | |
|---|---|---|---|---|---|
| 1 | STRBP | NM_001171137.1 | 1150-1250 | 98.2 | −0.27109 |
| 2 | PRKAR2B | NM_002736.2 | 1350-1450 | 92.9 | −0.22307 |
| 3 | E2F2 | NM_004091.2 | 3605-3705 | 80.5 | −0.19078 |
| 4 | LZTS1 | NM_021020.2 | 3970-4070 | 72.6 | −0.08932 |
| 5 | *CDC25A | NM_001789.2 | 690-790 | 72.6 | −0.20919 |
| 6 | TCF3 | NM_003200.2 | 4325-4425 | 69 | −0.08141 |
| 7 | *RANBP1 | NM_002882.2 | 380-480 | 58.4 | −0.51849 |
| 8 | *DLEU1 | NR_002605.1 | 173-273 | 54.9 | −0.23804 |
| 9 | *PAICS | NM_001079524.1 | 2604-2704 | 46.9 | −0.10796 |
| 10 | DNMT3B | NM_175850.1 | 1950-2050 | 45.1 | −0.10655 |
| 11 | *PPAT | NM_002703.3 | 1210-1310 | 44.2 | −0.02356 |
| 12 | *KIAA0101 | NM_014736.4 | 65-165 | 43.4 | −0.16103 |
| 13 | PYCR1 | NM_006907.2 | 513-613 | 38.1 | −0.17794 |
| 14 | CD10 | NM_000902.2 | 5059-5159 | 34.5 | −0.01825 |
| 15 | *NME1 | NM_000269.2 | 500-600 | 17.7 | −0.10337 |
| 16 | *FAM216A/ | NM_013300.2 | 722-822 | 7.1 | 0 |
| | C12ORF24 | | | | |
| 17 | BMP7 | NM_001719.1 | 525-625 | 0 | 0 |
| 18 | BCL2 | NM_000657.2 | 5-105 | 46 | 0.156014 |
| 19 | CD44 | NM_001001392.1 | 429-529 | 57.5 | 0.138693 |
| 20 | p50 (NFKB1) | NM_003998.2 | 1675-1775 | 72.6 | 0.093462 |
| 21 | BCL2A1 | NM_004049.2 | 80-180 | 100 | 0.134243 |

Table 4 - MYC Activity Classifier

| | | | | | |
|---|---|---|---|---|---|
| 1 | MYC | NM_002467.3 | 1610-1710 | 100 | −0.23085 |
| 2 | SRM | NM_003132.2 | 254-354 | 77.8 | −0.06241 |
| 3 | AKAP1 | NM_139275.1 | 2725-2825 | 73 | −0.39995 |
| 4 | *NME1 | NM_000269.2 | 500-600 | 72.2 | −0.21281 |
| 5 | FBL | NM_001436.3 | 883-983 | 71.4 | 0 |
| 6 | RFC3 | NM_002915.3 | 740-840 | 69.8 | −0.29743 |
| 7 | TCL1A | NM_001098725.1 | 867-967 | 66.7 | −0.06223 |
| 8 | POLD2 | NM_006230.1 | 505-605 | 61.9 | −0.02536 |
| 9 | *RANBP1 | NM_002882.2 | 380-480 | 61.9 | −0.29493 |
| 10 | GEMIN4 | NM_015721.2 | 1925-2025 | 60.3 | −0.21774 |
| 11 | MRPS34 | NM_023936.1 | 719-819 | 60.3 | −0.36331 |
| 12 | DHX33 | NM_001199699.1 | 2873-2973 | 59.5 | −0.37029 |
| 13 | PPRC1 | NM_015062.3 | 4640-4740 | 59.5 | 0 |
| 14 | *PPAT | NM_002703.3 | 1210-1310 | 57.9 | −0.02264 |
| 15 | *FAM216A/ | NM_013300.2 | 722-822 | 57.1 | −0.04518 |
| | C12ORF24 | | | | |
| 16 | *PAICS | NM_001079524.1 | 2604-2704 | 54.8 | −0.19101 |
| 17 | UCHL3 | NM_006002.3 | 375-475 | 53.2 | −0.46356 |
| 18 | NOLC1 | NM_004741.3 | 3405-3505 | 52.4 | −0.14156 |
| 19 | KIAA0226L | NM_025113.2 | 1071-1171 | 50.8 | 0 |
| 20 | PRMT1 | NM_001536.4 | 444-544 | 50.8 | 0 |
| 21 | LDHB | NM_001174097.1 | 1190-1290 | 49.2 | 0 |
| 22 | TRAP1 | NM_016292.2 | 1293-1393 | 47.6 | −0.2165 |
| 23 | AHCY | NM_000687.2 | 1805-1905 | 47.6 | 0 |
| 24 | LRP8 | NM_033300.2 | 1590-1690 | 45.2 | −0.05459 |
| 25 | EBNA1BP2 | NM_006824.2 | 318-418 | 43.7 | 0 |
| 26 | CDK4 | NM_000075.2 | 1055-1155 | 42.1 | 0 |
| 27 | ETFA | NM_001127716.1 | 630-730 | 41.3 | 0 |
| 28 | UCK2 | NM_012474.3 | 730-830 | 39.7 | 0 |
| 29 | CTPS | NM_001905.2 | 2570-2670 | 39.7 | −0.02031 |
| 30 | GOT2 | NM_002080.2 | 2145-2245 | 38.9 | −0.13632 |
| 31 | FAM211A/ | NR_027164.1 | 214-314 | 36.5 | 0 |
| | C17ORF76 | | | | |
| 32 | TMEM97 | NM_014573.2 | 2055-2155 | 36.5 | −0.12119 |
| 33 | RRS1 | NM_015169.3 | 1247-1347 | 36.5 | 0.024235 |
| 34 | DDX21 | NM_004728.2 | 685-785 | 34.9 | −0.02013 |
| 35 | PHB2 | NM_007273.3 | 1210-1310 | 34.1 | 0 |
| 36 | WDR3 | NM_006784.2 | 90-190 | 33.3 | 0.004247 |
| 37 | *KIAA0101 | NM_014736.4 | 65-165 | 31.7 | −0.20888 |
| 38 | FASN | NM_004104.4 | 5387-5487 | 31.7 | 0 |
| 39 | SAMD13 | NM_001010971.2 | 672-772 | 31 | 0 |
| 40 | *CDC25A | NM_001789.2 | 690-790 | 30.2 | −0.06062 |
| 41 | LYAR | NM_001145725.1 | 230-330 | 30.2 | −0.05712 |
| 42 | CAD | NM_004341.3 | 2380-2480 | 26.2 | 0 |
| 43 | APEX1 | NM_001641.2 | 727-827 | 25.4 | 0 |
| 44 | NOP2 | NM_001033714.1 | 1800-1900 | 22.2 | 0 |
| 45 | PHB | NM_002634.2 | 1270-1370 | 20.6 | −0.37328 |
| 46 | SSBP1 | NM_003143.1 | 235-335 | 19.8 | 0 |
| 47 | MRPS2 | NR_051968.1 | 1512-1612 | 19 | 0 |
| 48 | CIRH1A | NM_032830.2 | 84-184 | 17.5 | 0 |
| 49 | SLC16A1 | NM_003051.3 | 635-735 | 16.7 | 0 |
| 50 | BUB1B | NM_001211.4 | 835-935 | 15.1 | −0.01642 |
| 51 | APITD1 | NM_199294.2 | 950-1050 | 15.1 | 0 |
| 52 | NCL | NM_005381.2 | 1492-1592 | 9.5 | 0.10533 |
| 53 | *DLEU1 | NR_002605.1 | 173-273 | 7.9 | −0.23218 |
| 54 | PCDH9 | NM_020403.3 | 3580-3680 | 0 | −0.0167 |
| 55 | IGFBP2 | NM_000597.2 | 675-775 | 8.7 | 0.052917 |
| 56 | TDO2 | NM_005651.1 | 0-100 | 18.3 | 0.022456 |
| 57 | SLC12A8 | NM_024628.5 | 770-870 | 30.2 | 0.184233 |
| 58 | P2RY12 | NM_022788.3 | 230-330 | 40.5 | 0.134099 |
| 59 | TMEM119 | NM_181724.2 | 1490-1590 | 53.2 | 0.157568 |
| 60 | SHISA8 | NM_001207020.1 | 1111-1211 | 67.5 | 0.127419 |

*Genes indicated in bold type (n = 8) are included in both the Diagnostic and MYC
Transcriptional Activity Classifiers

Table 4 - Housekeeping Genes

| | Gene Symbol | Accession Number | Target Region (bps) |
|---|---|---|---|
| 1 | AAMP | NM_001087.3 | 1646-1746 |
| 2 | H3F3A | NM_002107.3 | 190-290 |
| 3 | HMBS | NM_000190.3 | 315-415 |
| 4 | KARS | NM_005548.2 | 1885-1985 |
| 5 | PSMB3 | NM_002795.2 | 340-440 |
| 6 | TUBB | NM_178014.2 | 320-420 |

Diagnostic and MYC Activity Scores

Elastic net models output class probabilities between 0 and 1 for each class (probability of class BL in the diagnostic classifier, and of class MYC IHC-High in the MYC Transcriptional Activity classifier), reflecting the confidence of a sample prediction. Prior to analysis, and in order to reflect the concept of a biological ‘intermediate’ between BL and DLBCL, we defined Diagnostic scores of >0.75 as representing ‘molecular BL’ (mBL), <0.25 ‘molecular DLBCL’ (mDLBCL), and 0.25-0.75 ‘molecularly intermediate’, respectively. MYC activity scores of “1” and “0” correspond to tumors with high MYC and low MYC (as modeled on IHC expression¹⁵) with greatest probability, respectively. During development of the MYC activity classifier 0.5 was optimized as the cut-off with the highest estimated accuracy to classify tumors with high and low MYC activity. Therefore 0.5 is used for statistics regarding the efficacy of the classifier and for correlation to clinical outcome.

Reproducibility of the Assay

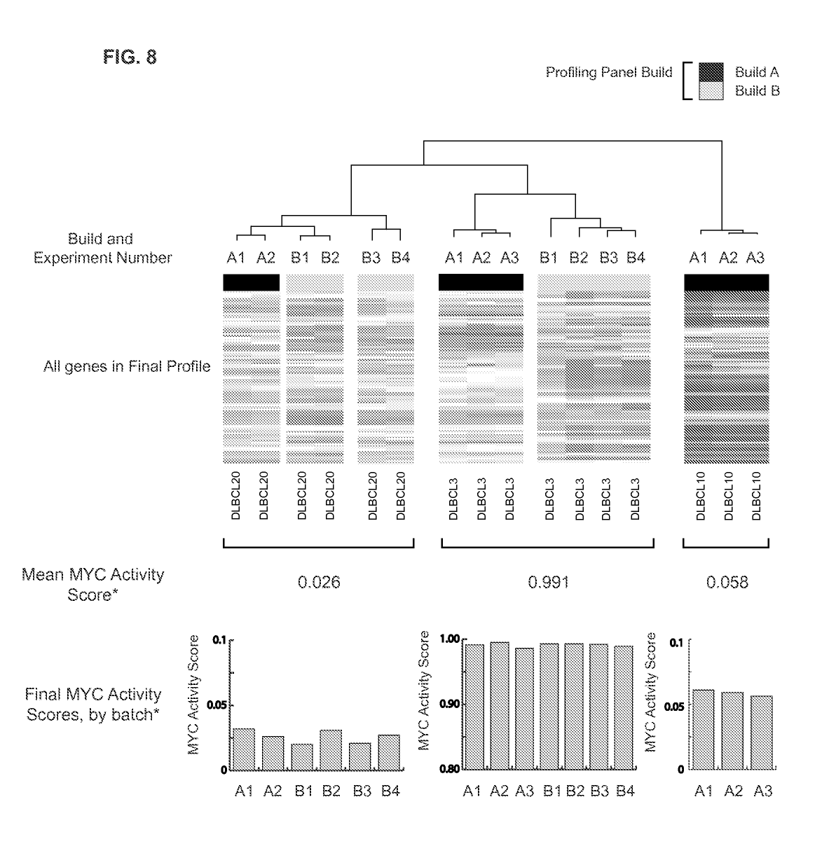

The test set and outcome series were profiled using 2 ‘builds’ (independently constructed probe sets) of the 80-gene profiling panel. The binding efficiency of probes varies between builds and therefore the final dataset was compiled by normalizing to both housekeepers and then between builds, using on the expression profiles of tumor RNA that were profiled on both. RNA from a subset of cases was profiled multiple times over the course of the study to determine the reproducibility of the assay (FIG. 8).

### Example 1

### Initial Data Profiling

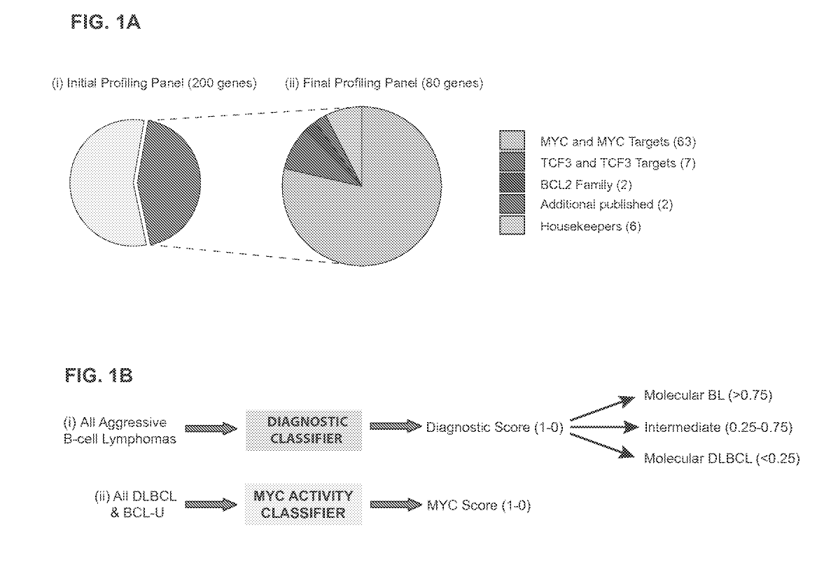

RNA was isolated from FFPE tissue corresponding to 41 aggressive B-cell lymphomas (training set) and was profiled using an initial panel of probes targeting 200 unique transcripts (FIG. 1 and Table 2). The resulting data were used to derive a pair of molecular classifiers, firstly to distinguish BL from DLBCL and secondly to distinguish high and low MYC activity in DLBCL using a parsimonious, 80-gene signature (FIG. 1 and Tables 3 and 4).

### Example 2

### Unsupervised Clustering of Targeted Expression Profiles of Select Lymphomas

Unsupervised clustering of the normalized expression data from the 200-gene signature segregated the training set tumors into distinct groups that showed a close correlation with the original pathological diagnoses of BL, DLBCL MYC IHC-High, and DLBCL MYC IHC-Low (FIG. 2). One DLBCL later failed classification quality control and was not used in subsequent analysis. One case, diagnosed as BL, clustered with DLBCL MYC IHC-High cases. Central review of this case confirmed that the tumor was originally diagnosed correctly. These data support the in silico methods used to develop the initial profiling panel and demonstrate the technical feasibility of the approach to broadly group aggressive lymphomas into clinically relevant categories.

### Example 3

### Performance of the Diagnostic Molecular Classifier on the Training and Test Sets

We tested the diagnostic molecular classifier against data derived from the training set in a LOO-CV, FIG. 3A). When ranked by the diagnostic classifier scores, these data largely recapitulated the results obtained from the original unsupervised clustering analysis using the 200-gene panel. Thirty-five of 41 (85%) cases classified as mBL or mDLBCL with high confidence and correctly matched the pathological diagnoses of BL or DLBCL, respectively (FIG. 3A, Table 5). Six cases had diagnostic scores of >0.25 and <0.75 and thus were not assigned to the categories of mBL or mDLBCL. Nevertheless, 3 of the “molecularly intermediate” cases had a pathological diagnosis of BL and 2 of these had a diagnostic score>0.5; three “molecularly intermediate” cases had a pathological diagnosis of DLBCL and 2 had a diagnostic score<0.5. We conclude that in our training cohort a 21-gene classifier can be used to distinguish the majority (85%) of pathological BL from DLBCL.

We next profiled and classified an independent test set of 55 cases that included 9 BL, 41 DLBCL, and 5 cases with the pathological diagnosis of BCL-U (FIG. 3B). Among the non-BLs were one genetic “single-hit” lymphoma (genetic SHL, with isolated MYC-translocation, tDLBCL1) and 8 genetic “double-hit” lymphomas (DHLs), all with MYC-translocations. Seven DHLs had coexistent BCL2-translocations and one DHL had a coexistent BCL6-translocation, tDHL1-8, (FIG. 3B).

The diagnostic classifier successfully segregated all pathological BL from all DLBCL (FIG. 3B, Table 5). Forty-six of 50 (92%) BL and DLBCL were classified with high confidence. Two BL and two DLBCL had intermediate diagnostic scores, but among these, the diagnostic scores for the BL were >0.5 and for the DLBCL≦0.5. The DLBCL with the highest diagnostic score (case tDLBCL1, score=0.5) was the genetic SHL. The diagnostic classifier demonstrated a sensitivity of 1.0 (95% CI 0.59-1.0) and specificity of 1.0 (95% CI 0.91-1.0) in the test set, for all tumors classified as mBL or mDLBCL (Table 5). We conclude that in our test cohort a 21-gene classifier can be used to distinguish the majority (92%) of pathological BL from DLBCL.

Molecular classification segregated subsets of non-BLs with the pathological diagnosis of BCL-U and/or genetic evidence for MYC-rearrangements into all three diagnostic categories (FIG. 3B). Three BCL-U/DHLs (tDHL1, tDHL2, tDHL3) had high diagnostic scores (0.90, 0.85, and 0.77, respectively) and classified as mBL. One DLBCL/SHL (tDLBCL1) and one BCL-U/DHL (tDHL4) had lower diagnostic scores (0.50 and 0.31, respectively) and classified as ‘molecularly intermediate’. Finally, one BCL-U/DHL (tDHL5) and three DLBCL/DHLs (tDHL6, tDHL7, tDHL8) had low diagnostic scores (0.12, 0.05, 0.02, and 0.015, respectively) and classified as mDLBCL. We conclude that the diagnostic molecular classifier reveals molecular heterogeneity among BCL-Us and DLBCLs with MYC-translocations.

TABLE 5
Performance Statistics of Molecular Classifiers

| | | | MYC Activity Classifier¶ | | | | |
|---|---|---|---|---|---|---|---|
| | Diagnostic | | | Training | | Test | |
| | Classifier* | | Training | (non- | Test | (non- | Outcome |
| | Training | Test | (All) | BL) | (All) | BL) | Series |
| Percentage of | 85% | 92% | 100% | 100% | 100% | 100% | 100% |
| cases | | | | | | | |
| classified | | | | | | | |
| Accuracy | 1 | 1 | 0.93 | 0.90 | 0.80 | 0.80 | 0.87 |
| Sensitivity | 1 | 1 | 0.92 | 0.85 | 0.77 | 0.69 | 0.75 |
| (95% CI) | (0.66-1.0) | (0.59-1.0) | (0.73-0.99) | (0.55-0.98) | (0.55-0.92) | (0.41-0.89) | (0.35-0.96) |
| Specificity | 1 | 1 | 0.94 | 0.94 | 0.83 | 0.86 | 0.90 |
| (95% CI) | (0.87-1.0) | (0.91-1.0) | (0.70-0.99) | (0.70-0.99) | (0.64-0.94) | (0.67-0.96) | (0.73-0.98) |
| Positive | 1 | 1 | 0.96 | 0.92 | 0.77 | 0.73 | 0.67 |
| Predictive | | | | | | | |
| Value (PPV) | | | | | | | |
| Negative | 1 | 1 | 0.88 | 0.88 | 0.83 | 0.83 | 0.93 |
| Predictive | | | | | | | |
| Value (NPV) | | | | | | | |

*Diagnostic Classifier: Only cases classified with high confidence (as mBL or mDLBCL) are included. The sensitivity refers to the ability of the test to identify pathological BL as molecular BL (‘mBL’).

MYC Activity Classifier: Only cases with matched MYC IHC and MYC Activity scores are included. The sensitivity refers to the ability of the test to identify tumors with high MYC IHC expression (>50%) as having MYC Activity score>0.5.

### Example 4

### Molecular and Histopathological Features of BCL-U/DHL

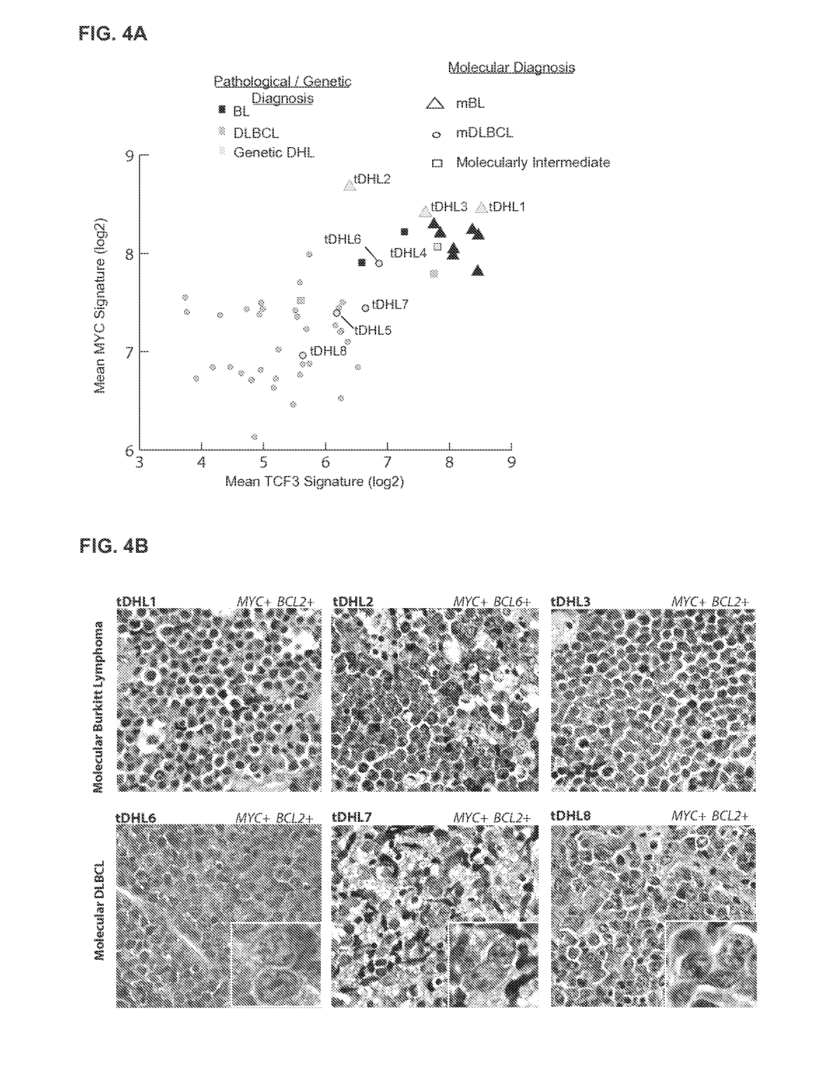

We next examined the molecular signatures and histopathology of the BCL-Us and DLBCLs with MYC-translocations in more detail (FIG. 4). BCL-U/DHLs classified as mBL expressed both TCF3-associated transcripts and MYC-associated transcripts at levels that were comparable to BL (FIG. 4A). DLBCL/DHLs classified as mDLBCL expressed TCF3-associated transcripts at low levels and MYC-associated transcripts at intermediate levels that were comparable to many DLBCLs lacking a MYC-translocation (FIG. 4A). Additional transcripts (BCL2, CD44, NFKB1 and BCL2A1) differentially expressed between BL and DLBCL, also showed differential expression among the DHLs, and with the TCF3 and MYC signatures, resulted in the final classification indicated in FIG. 3B.

Further review of the histopathology of the DHLs revealed distinct features between those that classify with high confidence as mBL and those that classified with high confidence as mDLBCL (FIG. 4B). Cases classified as mBL were composed of sheets of tightly packed, intermediate to large-sized cells with homogenous, round nuclei, and scant cytoplasm that resembles the morphological features of classic BL. In contrast, cases classified as mDLBCL were composed of large-sized lymphoid cells with marked pleomorphism and nuclear irregularity typical of DLBCL. We conclude that the final molecular classifications of DHLs are supported by multiple molecular signatures, and correlate with distinct histopathological characteristics.

### Example 5

### Performance of the MYC Activity Classifier on the Training and Test Sets

The MYC activity classifier was tested in the training cohort by LOO-CV. BLs were not used to build the classifier but, as expected, had very high MYC activity scores (FIG. 5A). In addition, all non-BLs with MYC-translocation had MYC activity scores>0.5. The sensitivity and specificity of the molecular classifier for identifying MYC IHC-High among all cases in the training set were 0.92 (95% CI 0.73-0.997) and 0.94 (95% CI 0.70-0.99), respectively (Table 5). Overall, the correlation between the optimized, molecular MYC activity score and MYC IHC score among non-BLs in the training set was high (Spearman r=0.80, p<0.0001, 95% CI 0.6-0.9, FIG. 5A).

We next applied the MYC activity classifier to expression data from the independent test set. Again, BL cases showed very high MYC activity scores (FIG. 5B). The sensitivity and specificity of the molecular classifier identifying MYC IHC-High among all cases were 0.77 (95% CI 0.55-0.92) and 0.83 (95% CI 0.64-0.94), respectively (Table 5). The correlation between the molecular MYC score and the MYC IHC score for the test set (non-BLs) was lower than for the LOO-CV of the training set, but with overlapping confidence intervals, thus preventing definitive comparison (Spearman r=0.66, p<0.0001, 95% CI 0.44-0.8).

Non-BLs with a MYC-translocation were expected to have upregulated MYC activity, and for 5 of 9 cases, tDHL1-4 and tDHL6, the MYC activity scores were high and comparable to those seen for BL (ranging from 0.98-1.00). There was a range of values among the remaining cases. For tDLBCL1 (genetic SHL) and tDHL5, the MYC activity scores were 0.63 and 0.60 and for tDHL7 and tDHL8, the scores were lower at 0.26 and 0.18 respectively. Non-BLs with MYC-translocations and high MYC activity scores had a pathological diagnosis of BCL-U whereas those with other MYC activity scores had a pathological diagnosis of DLBCL. We conclude that the MYC activity classifier captures a spectrum of MYC biological activity in BCL-U and DLBCL that shows good correlation with MYC IHC and reveals heterogeneity in MYC biological activity among non-BL with MYC translocations.

### Example 6

### Clinical Significance of the MYC Activity Score in DLBCL

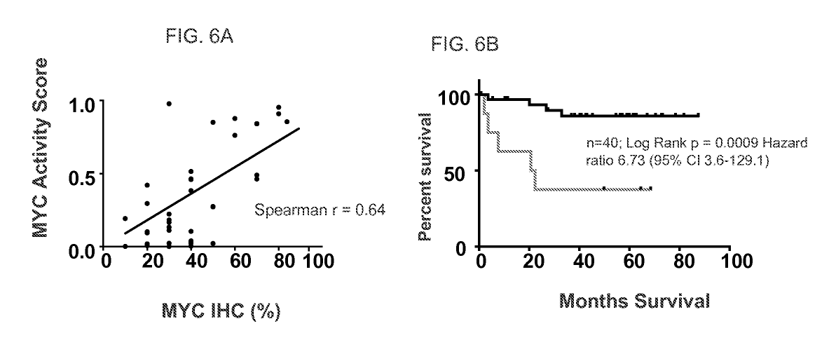

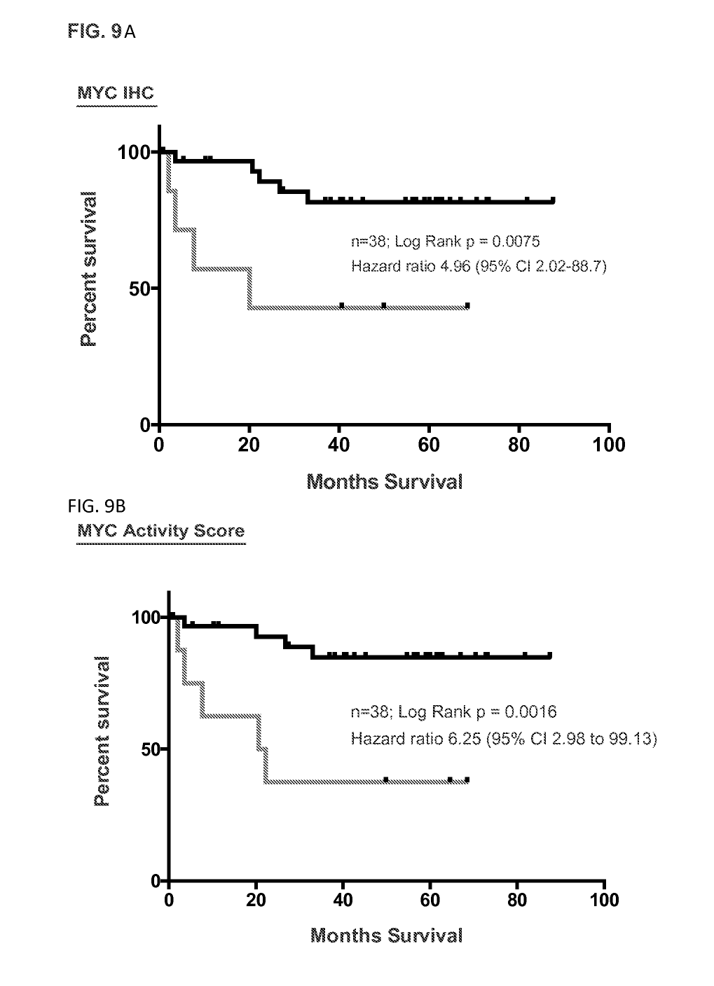

The MYC activity classifier was constructed in order to categorize aggressive B-cell lymphomas according to MYC biological activity, rather than to predict clinical outcome. The MYC activity scores showed good, but not perfect, correlation with MYC IHC scores in the training and test sets. Therefore, we wished to determine whether the results of the MYC classifier were sufficient to predict clinical outcome in a series for which MYC IHC has prognostic value¹⁵. DLBCLs with MYC activity scores in excess of the optimized classifier cut-point of 0.5 identified a patient population with inferior overall survival that was highly significant (nominal p=0.0009, log-rank test; hazard ratio=6.73, FIG. 6B). The correlation between MYC activity and MYC IHC scores being similar to the training and test sets, as expected (r=0.64, p<0.0001, 95% CI 0.4-0.8, FIG. 6A). We conclude that the MYC activity classifier, built upon MYC IHC data (FIG. 9), is capable of dividing patients into high-risk and low-risk categories.

### REFERENCES

1. Swerdlow S H, World Health Organization. WHO classification of tumours of haematopoietic and lymphoid tissues. 2008.
2. Magrath I, Adde M, Shad A, Venzon D, Seibel N, Gootenberg J, Neely J, Arndt C, Nieder M, Jaffe E, Wittes R A, Horak I D. Adults and children with small non-cleaved-cell lymphoma have a similar excellent outcome when treated with the same chemotherapy regimen. Journal of Clinical Oncology. 1996 Mar. 1; 14(3):925-34.
3. Habermann T M. Rituximab-CHOP Versus CHOP Alone or With Maintenance Rituximab in Older Patients With Diffuse Large B-Cell Lymphoma. Journal of Clinical Oncology. 2006 May 8; 24(19):3121-7.
4. Hecht J L, Aster J C. Molecular Biology of Burkitt's Lymphoma. Journal of Clinical Oncology. 2000 Nov. 1; 18(21):3707-21.
5. Schmitz R, Young R M, Ceribelli M, Jhavar S, Xiao W, Zhang M, Wright G, Shaffer A L, Hodson D J, Buras E, Liu X, Powell J, Yang Y, Xu W, Zhao H, Kohlhammer H, Rosenwald A, Kluin P, Müller-Hermelink H K, Ott G, Gascoyne R D, Connors J M, Rimsza L M, Campo E, Jaffe E S, Delabie J, Smeland E B, Ogwang M D, Reynolds S J, Fisher R I, Braziel R M, Tubbs R R, Cook J R, Weisenburger D D, Chan W C, Pittaluga S, Wilson W, Waldmann T A, Rowe M, Mbulaiteye S M, Rickinson A B, Staudt L M. Burkitt lymphoma pathogenesis and therapeutic targets from structural and functional genomics. Nature. 2012 Aug. 12; 490(7418):116-20.
6. Love C, Sun Z, Jima D, Li G, Zhang J, Miles R, Richards K L, Dunphy C H, Choi W W L, Srivastava G, Lugar P L, Rizzieri D A, Lagoo A S, Bernal-Mizrachi L, Mann K P, Flowers C R, Naresh K N, Evens A M, Chadburn A, Gordon L I, Czader M B, Gill J I, Hsi E D, Greenough A, Moffitt A B, McKinney M, Banerjee A, Grubor V, Levy S, Dunson D B, Dave S S. The genetic landscape of mutations in Burkitt lymphoma. Nat Genet. 2012 Nov. 11; 44(12):1321-5.
7. Zhang J, Grubor V, Love C L, Banerjee A, Richards K L, Mieczkowski P A, Dunphy C, Choi W, Au W Y, Srivastava G, Lugar P L, Rizzieri D A, Lagoo A S, Bernal-Mizrachi L, Mann K P, Flowers C, Naresh K, Evens A, Gordon L I, Czader M, Gill J I, Hsi E D, Liu Q, Fan A, Walsh K, Jima D, Smith L L, Johnson A J, Byrd J C, Luftig M A, Ni T, Zhu J, Chadburn A, Levy S, Dunson D, Dave S S. Genetic heterogeneity of diffuse large B-cell lymphoma. Proc Natl Acad Sci USA. 2013 Jan. 22; 110(4):1398-403.
8. Morin R D, Mungall K, Pleasance E, Mungall A J, Goya R, Huff R, Scott D W, Ding J, Roth A, Chiu R, Corbett R D, Chan F C, Mendez-Lago M, Trinh D L, Bolger-Munro M, Taylor G, Hadj Khodabakhshi A, Ben-Neriah S, Pon J, Meissner B, Woolcock B, Farnoud N, Rogic S, Lim E, Johnson N A, Shah S, Jones S, Steidl C, Holt R, Birol I, Moore R, Connors J M, Gascoyne R D, Marra M A. Mutational and structural analysis of diffuse large B-cell lymphoma using whole genome sequencing. Blood. 2013 May 22.
9. Lohr J G, Stojanov P, Lawrence M S, Auclair D, Chapuy B, Sougnez C, Cruz-Gordillo P, Knoechel B, Asmann Y W, Slager S L. Discovery and prioritization of somatic mutations in diffuse large B-cell lymphoma (DLBCL) by whole-exome sequencing. Proc Natl Acad Sci USA. 2012; 109(10):3879-84.
10. Savage K J, Johnson N A, Ben-Neriah S, Connors J M, Sehn L H, Farinha P, Horsman D E, Gascoyne R D. MYC gene rearrangements are associated with a poor prognosis in diffuse large B-cell lymphoma patients treated with R-CHOP chemotherapy. Blood. 2009 Oct. 22; 114(17):3533-7.
11. Barrans S, Crouch S, Smith A, Turner K, Owen R, Patmore R, Roman E, Jack A. Rearrangement of MYC Is Associated With Poor Prognosis in Patients With Diffuse Large B-Cell Lymphoma Treated in the Era of Rituximab. Journal of Clinical Oncology. 2010 Jul. 8; 28(20):3360-5.
12. Dave S S, Fu K, Wright G W, Lam L T, Kluin P, Boerma E-J, Greiner T C, Weisenburger D D, Rosenwald A, Ott G. Molecular diagnosis of Burkitt's lymphoma. New England Journal of Medicine. 2006; 354(23):2431-42.
13. Hummel M, Bentink S, Berger H, Klapper W, Wessendorf S, Barth T F, Bernd H-W, Cogliatti S B, Dierlamm J, Feller A C. A biologic definition of Burkitt's lymphoma from transcriptional and genomic profiling. New England Journal of Medicine. 2006; 354(23):2419-30.
14. Johnson N A, Slack G W, Savage K J, Connors J M, Ben-Neriah S, Rogic S, Scott D W, Tan K L, Steidl C, Sehn L H, Chan W C, Iqbal J, Meyer P N, Lenz G, Wright G, Rimsza L M, Valentino C, Brunhoeber P, Grogan T M, Braziel R M, Cook J R, Tubbs R R, Weisenburger D D, Campo E, Rosenwald A, Ott G, Delabie J, Holcroft C, Jaffe E S, Staudt L M, Gascoyne R D. Concurrent expression of MYC and BCL2 in diffuse large B-cell lymphoma treated with rituximab plus cyclophosphamide, doxorubicin, vincristine, and prednisone. Journal of Clinical Oncology. 2012 Oct. 1; 30(28):3452-9.
15. Kluk M J, Chapuy B, Sinha P, Roy A, Cin P D, Neuberg D S, Monti S, Pinkus G S, Shipp M A, Rodig S J. Immunohistochemical Detection of MYC-driven Diffuse Large B-Cell Lymphomas. Zhang L, editor. PLoS ONE. 2012 Apr. 12; 7(4):e33813.
16. Zhou K, Xu D, Cao Y, Wang J, Yang Y, Huang M. C-MYC Aberrations as Prognostic Factors in Diffuse Large B-cell Lymphoma: A Meta-Analysis of Epidemiological Studies. Ariga H, editor. PLoS ONE. 2014; 9(4):e95020.
17. Cook J R, Goldman B, Tubbs R R. Clinical Significance of MYC Expression and/or “High-grade” Morphology in Non-Burkitt, Diffuse Aggressive B-cell Lymphomas: A SWOG S9704 Correlative Study. The American Journal of Surgical Pathology. 2014.
18. Perry A M, Alvarado-Bernal Y, Laurini J A, Smith L M, Slack G W, Tan K L, Sehn L H, Fu K, Aoun P, Greiner T C, Chan W C, Bierman P J, Bociek R G, Armitage J O, Vose J M, Gascoyne R D, Weisenburger D D. MYC and BCL2 protein expression predicts survival in patients with diffuse large B-cell lymphoma treated with rituximab. British Journal of Haematology. 2014; 165(3):382-91.
19. Green T M, Young K H, Visco C, Xu-Monette Z Y, Orazi A, Go R S, Nielsen O, Gadeberg O V, Mounts-Andersen T, Frederiksen M, Pedersen L M, Moller M B. Immunohistochemical Double-Hit Score Is a Strong Predictor of Outcome in Patients With Diffuse Large B-Cell Lymphoma Treated With Rituximab Plus Cyclophosphamide, Doxorubicin, Vincristine, and Prednisone. Journal of Clinical Oncology. 2012 Sep. 28; 30(28):3460-7.
20. Horn H, Ziepert M, Becher C, Barth TFE, Bernd H W, Feller A C, Klapper W, Hummel M, Stein H, Hansmann M L, Schmelter C, Moller P, Cogliatti S, Pfreundschuh M, Schmitz N, Trumper L, Siebert R, Loeffler M, Rosenwald A, Ott G, for the German High-Grade Non-Hodgkin Lymphoma Study Group. MYC status in concert with BCL2 and BCL6 expression predicts outcome in diffuse large B-cell lymphoma. Blood. 2013 Mar. 21; 121(12):2253-63.
21. Hu S, Xu-Monette Z Y, Tzankov A, Green T, Wu L, Balasubramanyam A, Liu W M, Visco C, Li Y, Miranda R N, Montes-Moreno S, Dybkaer K, Chiu A, Orazi A, Zu Y, Bhagat G, Richards K L, Hsi E D, Choi WWL, Zhao X, van Krieken J H, Huang Q, Huh J, Ai W, Ponzoni M, Ferreri A J M, Zhou F, Slack G W, Gascoyne R D, Tu M, Variakojis D, Chen W, Go R S, Pins M A, Moller M B, Medeiros L J, Young K H. MYC/BCL2 protein coexpression contributes to the inferior survival of activated B-cell subtype of diffuse large B-cell lymphoma and demonstrates high-risk gene expression signatures: a report from The International DLBCL Rituximab-CHOP Consortium Program. Blood. 2013 May 16; 121(20):4021-31.
22. de Jong D, Rosenwald A, Chhanabhai M, Gaulard P, Klapper W, Lee A, Sander B, Thorns C, Campo E, Molina T, Norton A, Hagenbeek A, Horning S, Lister A, Raemaekers J, Gascoyne R D, Salles G, Weller E. Immunohistochemical Prognostic Markers in Diffuse Large B-Cell Lymphoma: Validation of Tissue Microarray As a Prerequisite for Broad Clinical Applications—A Study From the Lunenburg Lymphoma Biomarker Consortium. Journal of Clinical Oncology. 2007 Jan. 16; 25(7):805-12.
23. Rimsza L M, Wright G, Schwartz M, Chan W C, Jaffe E S, Gascoyne R D, Campo E, Rosenwald A, Ott G, Cook J R, Tubbs R R, Braziel R M, Delabie J, Miller T P, Staudt L M. Accurate Classification of Diffuse Large B-Cell Lymphoma into Germinal Center and Activated B-Cell Subtypes Using a Nuclease Protection Assay on Formalin-Fixed, Paraffin-Embedded Tissues. Clinical Cancer Research. 2011 Jun. 1; 17(11):3727-32.
24. Scott D W, Wright G W, Williams P M, Lih C-J, Walsh W, Jaffe E S, Rosenwald A, Campo E, Chan W C, Connors J M, Smeland E B, Mottok A, Braziel R M, Ott G, Delabie J, Tubbs R R, Cook J R, Weisenburger D D, Greiner T C, Glinsmann-Gibson B J, Fu K, Staudt L M, Gascoyne R D, Rimsza L M. Determining cell-of-origin subtypes of diffuse large B-cell lymphoma using gene expression in formalin-fixed paraffin embedded tissue. Blood. 2014 Jan. 7; 123(8):1214-7.
25. Masqué-Soler N, Szczepanowski M, Kohler C W, Spang R, Klapper W. Molecular classification of mature aggressive B-cell lymphoma using digital multiplexed gene expression on formalin-fixed paraffin-embedded biopsy specimens. Blood. 2013 Sep. 12; 122(11):1985-6.
26. Linton K, Howarth C, Wappett M, Newton G, Lachel C, Iqbal J, Pepper S, Byers R, Chan W J, Radford J. Microarray gene expression analysis of fixed archival tissue permits molecular classification and identification of potential therapeutic targets in diffuse large B-cell lymphoma. J Mol Diagn. 2012 May; 14(3):223-32.
27. Alizadeh A A, Eisen M B, Davis R E, Ma C, Lossos I S, Rosenwald A, Boldrick J C, Sabet H, Tran T, Yu X, Powell J I, Yang L, Marti G E, Moore T, Hudson J, Lu L, Lewis D B, Tibshirani R, Sherlock G, Chan W C, Greiner T C, Weisenburger D D, Armitage J O, Warnke R, Levy R, Wilson W, Grever M R, Byrd J C, Botstein D, Brown P O, Staudt L M. Distinct types of diffuse large B-cell lymphoma identified by gene expression profiling. Nature. 2000 Feb. 3; 403(6769):503-11.
28. Hans C P. Confirmation of the molecular classification of diffuse large B-cell lymphoma by immunohistochemistry using a tissue microarray. Blood. 2004 Jan. 1; 103(1):275-82.
29. Bogusz A M, Baxter R H G, Currie T, Sinha P, Sohani A R, Kutok J L, Rodig S J. Quantitative Immunofluorescence Reveals the Signature of Active B-cell Receptor Signaling in Diffuse Large B-cell Lymphoma. Clinical Cancer Research. 2012 Nov. 14; 18(22):6122-35.
30. Geiss G K, Bumgarner R E, Birditt B, Dahl T, Dowidar N, Dunaway D L, Fell H P, Ferree S, George R D, Grogan T, James J J, Maysuria M, Mitton J D, Oliveri P, Osborn J L, Peng T, Ratcliffe A L, Webster P J, Davidson E H, Hood L. Direct multiplexed measurement of gene expression with color-coded probe pairs. Nat Biotechnol. 2008 Feb. 17; 26(3):317-25.
31. Zeller K I, Jegga A G, Aronow B J, O'Donnell K A, Dang C V. An integrated database of genes responsive to the Myc oncogenic transcription factor: identification of direct genomic targets. Genome Biol. 2003; 4(10):R69.
32. Mori S, Rempel R E, Chang J T, Yao G, Lagoo A S, Potti A, Bild A, Nevins J R. Utilization of Pathway Signatures to Reveal Distinct Types of B Lymphoma in the E-myc Model and Human Diffuse Large B-Cell Lymphoma. Cancer Research. 2008 Oct. 15; 68(20):8525-34.
33. Schuhmacher M, Kohlhuber F, Hölzel M, Kaiser C, Burtscher H, Jarsch M, Bornkamm G W, Laux G, Polack A, Weidle U H, Eick D. The transcriptional program of a human B cell line in response to Myc. Nucleic Acids Research. 2001 Jan. 15; 29(2):397-406.
34. Kim Y H, Girard L, Giacomini C P, Wang P, Hernandez-Boussard T, Tibshirani R, Minna J D, Pollack J R. Combined microarray analysis of small cell lung cancer reveals altered apoptotic balance and distinct expression signatures of MYC family gene amplification. Oncogene. 2005 Aug. 22; 25(1):130-8.
35. Chapuy B, McKeown M R, Lin C Y, Monti S. Discovery and Characterization of Super-Enhancer-Associated Dependencies in Diffuse Large B Cell Lymphoma. Cancer Cell. 2013; 24(6):777-90.
36. Schlosser I, Hölzel M, Hoffmann R, Burtscher H, Kohlhuber F, Schuhmacher M, Chapman R, Weidle U H, Eick D. Dissection of transcriptional programmes in response to serum and c-Myc in a human B-cell line. Oncogene. 2004 Nov. 1; 24(3):520-4.
37. Yu D, Cozma D, Park A, Thomas-Tikhonenko A. Functional Validation of Genes Implicated in Lymphomagenesis: An in Vivo Selection Assay Using a Myc-Induced B-Cell Tumor. Annals of the New York Academy of Sciences. 2005; 1059(1):145-59.
38. Monti S, Chapuy B, Takeyama K, Rodig S J, Hao Y, Yeda K T, Inguilizian H, Mermel C, Currie T, Dogan A, Kutok J L, Beroukhim R, Neuberg D, Habermann T M, Getz G, Kung A L, Golub T R, Shipp M A. Integrative Analysis Reveals an Outcome-Associated and Targetable Pattern of p53 and Cell Cycle Deregulation in Diffuse Large B Cell Lymphoma. Cancer Cell. 2012 Sep. 11; 22(3):359-72.
39. Zou H, Hastie T. Regularization and variable selection via the elastic net—Journal of the Royal Statistical Society: Series B (Statistical Methodology). Journal of the Royal Statistical Society. 2005.
40. Tibshirani R, Hastie T, Narasimhan B, Chu G. Diagnosis of multiple cancer types by shrunken centroids of gene expression. Proc Natl Acad Sci USA. 2002 May 14; 99(10):6567-72.
41. Breiman L. Random Forests—Springer. Machine learning. 2001.
42. Salaverria I, Siebert R. The Gray Zone Between Burkitt's Lymphoma and Diffuse Large B-Cell Lymphoma From a Genetics Perspective. Journal of Clinical Oncology. 2011 May 10; 29(14):1835-43.
43. Snuderl M, Kolman O K, Chen Y-B, Hsu J J, Ackerman A M, Cin P D, Ferry J A, Harris N L, Hasserjian R P, Zukerberg L R, Abramson J S, Hochberg E P, Lee H, Lee A I, Toomey C E, Sohani A R. B-cell Lymphomas With Concurrent IGH-BCL2 and MYC Rearrangements Are Aggressive Neoplasms With Clinical and Pathologic Features Distinct From Burkitt Lymphoma and Diffuse Large B-cell Lymphoma. The American Journal of Surgical Pathology. 2010 March; 34(3):327-40.
44. Aukema S M, Kreuz M, Kohler C W, Rosolowski M, Hasenclever D, Hummel M, Küppers R, Lenze D, Ott G, Pott C, Richter J, Rosenwald A, Szczepanowski M, Schwaenen C, Stein H, Trautmann H, Wessendorf S, Trümper L, Loeffler M, Spang R, Kluin P M, Klapper W, Siebert R. Biologic characterization of adult MYC-translocation positive mature B-cell lymphomas other than molecular Burkitt lymphoma. Haematologica. 2013 Oct. 31; 99(4):726-35.
45. Gebauer N, Bernard V, Feller A C, Merz H. ID3 Mutations Are Recurrent Events in Double-hit B-Cell Lymphomas. Anticancer Res. 2013 November; 33(11):4771-8.
46. Gebauer N, Bernard V, Gebauer W, Thorns C, Feller A C, Merz H. TP53 Mutations are frequent events in Double-Hit B-cell lymphomas with MYC and BCL2 but not MYC and BCL6 translocations. Leukemia & Lymphoma. 2014 Mar. 29; 1-15.
47. Johnson N A, Savage K J, Ludkovski O, Ben-Neriah S, Woods R, Steidl C, Dyer M J S, Siebert R, Kuruvilla J, Klasa R, Connors J M, Gascoyne R D, Horsman D E. Lymphomas with concurrent BCL2 and MYC translocations: the critical factors associated with survival. Blood. 2009 Sep. 10; 114(11):2273-9.
48. Friedberg J W. Double-hit diffuse large B-cell lymphoma. Journal of Clinical Oncology. 2012 Oct. 1; 30(28):3439-43.
49. Valera A, Lopez-Guillermo A, Cardesa-Salzmann T, Climent F, Gonzalez-Barca E, Mercadal S, Espinosa I, Novelli S, Briones J, Mate J L, Salamero O, Sancho J M, Arenillas L, Serrano S, Erill N, Martinez D, Castillo P, Rovira J, Martinez A, Campo E, Colomo L. MYC protein expression and genetic alterations have prognostic impact in diffuse large B-cell lymphoma treated with immunochemotherapy. Haematologica. 2013 May 28; 98(10):1554-62.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of diagnosing a subject who has a B-cell lymphoma as having Burkitt lymphoma (BL) or diffuse large B-cell lymphoma (DLBCL), the method comprising:
obtaining a sample comprising cells from the B-cell lymphoma in a subject;
determining levels of mRNA for diagnostic signature genes in the cells, wherein the diagnostic signature genes comprise STRBP, PRKAR2B, E2F2, LZTS1, CDC25A, TCF3, RANBP1, DLEU1, PAICS, DNMT3B, PPAT, KIAA0101, PYCR1, CD10, NME1, FAM216A/C12ORF24, BMP7, BCL2, CD44, p50 (NFKB1), and BCL2A;
calculating a diagnostic score based on the mRNA levels; and
diagnosing DLBCL when the diagnostic score is below a first threshold, diagnosing BL when the diagnostic scores is above a second threshold that is higher than the first threshold, and diagnosing intermediate B-cell lymphoma when the diagnostic score is between the first and second thresholds.

2. A method of treating a subject who has a B-cell lymphoma, the method comprising:
obtaining a sample comprising cells from a B-cell lymphoma in a subject;
determining levels of mRNA for MYC activity signature genes in the cells, wherein the MYC activity signature genes comprise MYC, SRM, AKAP1, NME1, FBL, RFC3, TCL1A, POLD2, RANBP1, GEMIN4, MRPS34, DHX33, PPRC1, PPAT, FAM216A/C12ORF24, PAICS, UCHL3, NOLC1, KIAA0226L, PRMT1, LDHB, TRAP1, AHCY, LRP8, EBNA1BP2, CDK4, ETFA, UCK2, CTPS, GOT2, FAM211A/C17ORF76, TMEM97, RRS1, DDX21, PHB2, WDR3, KIAA0101, FASN, SAMD13, CDC25A, LYAR, CAD, APEX1, NOP2, PHB, SSBP1, MRPS2, CIRH1A, SLC16A1, BUB1B, APITD1, NCL, DLEU1, PCDH9, IGFBP2, TDO2, SLC12A8, P2RY12, TMEM119, SHISA8, and SLAMF1;
calculating a MYC activity score based on the mRNA levels;
comparing the MYC activity score to a threshold level; and
administering a treatment to a subject who has a MYC activity score below the threshold level.

3. (canceled)

4. (canceled)

5. The method of claim 2, wherein the treatment is the R-CHOP (rituximab, cyclophosphamide, doxorubicin, vincristine, and prednisone) regimen.

6. A method of selecting, excluding or stratifying a subject for a clinical trial, the method comprising one or both of:
(i) determining a diagnostic score for the subject by obtaining a sample comprising cells from the B-cell lymphoma in the subject;
determining levels of mRNA for diagnostic signature genes in the cells, wherein the diagnostic signature genes comprise STRBP, PRKAR2B, E2F2, LZTS1, CDC25A, TCF3, RANBP1, DLEU1, PAICS, DNMT3B, PPAT, KIAA0101, PYCR1, CD10, NME1, FAM216A/C12ORF24, BMP7, BCL2, CD44, p50 (NFKB1), and BCL2A;
calculating a diagnostic score based on the mRNA levels; and/or
(ii) determining a MYC activity score method for the subject by obtaining a sample comprising cells from a B-cell lymphoma in a subject;
determining levels of mRNA for MYC activity signature genes in the cells, wherein the diagnostic signature genes comprise MYC, SRM, AKAP1, NME1, FBL, RFC3, TCL1A, POLD2, RANBP1, GEMIN4, MRPS34, DHX33, PPRC1, PPAT, FAM216A/C12ORF24, PAICS, UCHL3, NOLC1, KIAA0226L, PRMT1, LDHB, TRAP1, AHCY, LRP8, EBNA1BP2, CDK4, ETFA, UCK2, CTPS, GOT2, FAM211A/C17ORF76, TMEM97, RRS1, DDX21, PHB2, WDR3, KIAA0101, FASN, SAMD13, CDC25A, LYAR, CAD, APEX1, NOP2, PHB, SSBP1, MRPS2, CIRH1A, SLC16A1, BUB1B, APITD1, NCL, DLEU1, PCDH9, IGFBP2, TDO2, SLC12A8, P2RY12, TMEM119, SHISA8, and SLAMF1; and
calculating a MYC activity score based on the mRNA levels; and
predicting response to treatment based on the MYC activity score,
and selecting, excluding or stratifying the subject based on the MYC activity score and/or the diagnostic score.

7. The method of claim 1, comprising determining levels of one or more housekeeping genes, selected from the group consisting of AAMP, H3F3A, HMBS, KARS, PSMB3, and TUBB.

8. The method of claim 7, comprising normalizing expression levels of the signature genes to the levels of the housekeeping genes.

9. The method of claim 1, wherein determining a diagnostic score comprises applying a logistic regression model with elastic net regularization to the mRNA levels.

10. (canceled)

11. The method of claim 9, wherein the mRNA levels are weighted.

12. The method of claim 11, wherein the mRNA levels are weighted using the Gene weights shown in the following Table:

| Gene Symbol | Gene weights |
|---|---|
| STRBP | −0.27109 |
| PRKAR2B | −0.22307 |
| E2F2 | −0.19078 |
| LZTS1 | −0.08932 |
| *CDC25A | −0.20919 |
| TCF3 | −0.08141 |
| *RANBP1 | −0.51849 |
| *DLEU1 | −0.23804 |
| *PAICS | −0.10796 |
| DNMT3B | −0.10655 |
| *PPAT | −0.02356 |
| *KIAA0101 | −0.16103 |
| PYCR1 | −0.17794 |
| CD10 | −0.01825 |
| *NME1 | −0.10337 |
| *FAM216A/C12ORF24 | 0 |
| BMP7 | 0 |
| BCL2 | 0.156014 |
| CD44 | 0.138693 |
| p50 (NFKB1) | 0.093462 |
| BCL2A1 | 0.134243 |

13. The method of claim 1, wherein the score is calculated using a suitably programmed computing device.

14. The method of claim 1, wherein the score is calculated using a logistic regression function.

15. The method of claim 14, wherein the logistic regression function is:
$$p = \frac{1}{1 + {e}^{- ( {β}_{0} + {β}_{1} {x}_{1} + {β}_{2} {x}_{2} + … + {β}_{n} {x}_{n} )}} ,$$
Where p is the probability that a patient belongs to a certain class,
β₀ represents the intercept of the logistic regression model,
β_{1 . . . n} are the gene weights as shown in the following Table:

| Gene Symbol | Gene weights |
|---|---|
| STRBP | −0.27109 |
| PRKAR2B | −0.22307 |
| E2F2 | −0.19078 |
| LZTS1 | −0.08932 |
| *CDC25A | −0.20919 |
| TCF3 | −0.08141 |
| *RANBP1 | −0.51849 |
| *DLEU1 | −0.23804 |
| *PAICS | −0.10796 |
| DNMT3B | −0.10655 |
| *PPAT | −0.02356 |
| *KIAA0101 | −0.16103 |
| PYCR1 | −0.17794 |
| CD10 | −0.01825 |
| *NME1 | −0.10337 |
| *FAM216A/C12ORF24 | 0 |
| BMP7 | 0 |
| BCL2 | 0.156014 |
| CD44 | 0.138693 |
| p50 (NFKB1) | 0.093462 |
| BCL2A1 | 0.134243 |

and x_{1 . . . n} represent the gene expression values derived from a patient sample.

16. The method of claim 2, comprising determining levels of one or more housekeeping genes, selected from the group consisting of AAMP, H3F3A, HMBS, KARS, PSMB3, and TUBB.

17. The method of claim 16, comprising normalizing expression levels of the signature genes to the levels of the housekeeping genes.

18. The method of claim 2, wherein determining a MYC activity score comprises applying a logistic regression model with elastic net regularization to the mRNA levels.

19. The method of claim 18, wherein the mRNA levels are weighted.

20. The method of claim 19, wherein the mRNA levels are weighted using the weights shown in the following Table:

| Gene Symbol | Gene weights |
|---|---|
| MYC | −0.23085 |
| SRM | −0.06241 |
| AKAP1 | −0.39995 |
| *NME1 | −0.21281 |
| FBL | 0 |
| RFC3 | −0.29743 |
| TCL1A | −0.06223 |
| POLD2 | −0.02536 |
| *RANBP1 | −0.29493 |
| GEMIN4 | −0.21774 |
| MRPS34 | −0.36331 |
| DHX33 | −0.37029 |
| PPRC1 | 0 |
| *PPAT | −0.02264 |
| *FAM216A/C12ORF24 | −0.04518 |
| *PAICS | −0.19101 |
| UCHL3 | −0.46356 |
| NOLC1 | −0.14156 |
| KIAA0226L | 0 |
| PRMT1 | 0 |
| LDHB | 0 |
| TRAP1 | −0.2165 |
| AHCY | 0 |
| LRP8 | −0.05459 |
| EBNA1BP2 | 0 |
| CDK4 | 0 |
| ETFA | 0 |
| UCK2 | 0 |
| CTPS | −0.02031 |
| GOT2 | −0.13632 |
| FAM211A/ | 0 |
| C17ORF76 | |
| TMEM97 | −0.12119 |
| RRS1 | 0.024235 |
| DDX21 | −0.02013 |
| PHB2 | 0 |
| WDR3 | 0.004247 |
| *KIAA0101 | −0.20888 |
| FASN | 0 |
| SAMD13 | 0 |
| *CDC25A | −0.06062 |
| LYAR | −0.05712 |
| CAD | 0 |
| APEX1 | 0 |
| NOP2 | 0 |
| PHB | −0.37328 |
| SSBP1 | 0 |
| MRPS2 | 0 |
| CIRH1A | 0 |
| SLC16A1 | 0 |
| BUB1B | −0.01642 |
| APITD1 | 0 |
| NCL | 0.10533 |
| *DLEU1 | −0.23218 |
| PCDH9 | −0.0167 |
| IGFBP2 | 0.052917 |
| TDO2 | 0.022456 |
| SLC12A8 | 0.184233 |
| P2RY12 | 0.134099 |
| TMEM119 | 0.157568 |
| SHISA8 | 0.127419 |

21. The method of claim 2, wherein the score is calculated using a suitably programmed computing device.

22. The method of claim 2, wherein the score is calculated using a logistic regression function.

23. The method of claim 22, wherein the logistic regression function is:
$$p = \frac{1}{1 + {e}^{- ( {β}_{0} + {β}_{1} {x}_{1} + {β}_{2} {x}_{2} + … + {β}_{n} {x}_{n} )}} ,$$
Where p is the probability that a patient belongs to a certain class,
β₀ represents the intercept of the logistic regression model,
β_{1 . . . n} are the gene weights as shown in the following Table:

| Gene Symbol | Gene weights |
|---|---|
| MYC | −0.23085 |
| SRM | −0.06241 |
| AKAP1 | −0.39995 |
| *NME1 | −0.21281 |
| FBL | 0 |
| RFC3 | −0.29743 |
| TCL1A | −0.06223 |
| POLD2 | −0.02536 |
| *RANBP1 | −0.29493 |
| GEMIN4 | −0.21774 |
| MRPS34 | −0.36331 |
| DHX33 | −0.37029 |
| PPRC1 | 0 |
| *PPAT | −0.02264 |
| *FAM216A/C12ORF24 | −0.04518 |
| *PAICS | −0.19101 |
| UCHL3 | −0.46356 |
| NOLC1 | −0.14156 |
| KIAA0226L | 0 |
| PRMT1 | 0 |
| LDHB | 0 |
| TRAP1 | −0.2165 |
| AHCY | 0 |
| LRP8 | −0.05459 |
| EBNA1BP2 | 0 |
| CDK4 | 0 |
| ETFA | 0 |
| UCK2 | 0 |
| CTPS | −0.02031 |
| GOT2 | −0.13632 |
| FAM211A/ | 0 |
| C17ORF76 | |
| TMEM97 | −0.12119 |
| RRS1 | 0.024235 |
| DDX21 | −0.02013 |
| PHB2 | 0 |
| WDR3 | 0.004247 |
| *KIAA0101 | −0.20888 |
| FASN | 0 |
| SAMD13 | 0 |
| *CDC25A | −0.06062 |
| LYAR | −0.05712 |
| CAD | 0 |
| APEX1 | 0 |
| NOP2 | 0 |
| PHB | −0.37328 |
| SSBP1 | 0 |
| MRPS2 | 0 |
| CIRH1A | 0 |
| SLC16A1 | 0 |
| BUB1B | −0.01642 |
| APITD1 | 0 |
| NCL | 0.10533 |
| *DLEU1 | −0.23218 |
| PCDH9 | −0.0167 |
| IGFBP2 | 0.052917 |
| TDO2 | 0.022456 |
| SLC12A8 | 0.184233 |
| P2RY12 | 0.134099 |
| TMEM119 | 0.157568 |
| SHISA8 | 0.127419 |

and x_{1 . . . n} represent the gene expression values derived from a patient sample.
